(19) United States
(12) Patent Application Publication
(10) Pub. No.: US 2015/0320503 A1
(43) Pub. Date: Nov. 12, 2015

(54) STERILIZATION ASSEMBLY AND METHODS OF USE

(71) Applicant: Vatche BEZDIKIAN, Santa Rosa, CA (US)

(72) Inventor: Vatche BEZDIKIAN, Santa Rosa, CA (US)

(21) Appl. No.: 14/685,360

(22) Filed: Apr. 13, 2015

Related U.S. Application Data
(60) Provisional application No. 61/989,318, filed on May 6, 2014.
(60) Provisional application No. 62/083,777, filed on Nov. 24, 2014.

(51) Int. Cl. A61B 19/00; A61L 2/23; A61L 2/18; A61L 2/02

(52) U.S. Cl. CPC A61B 19/34; A61L 2/02; A61L 2/23; A61L 2/186; A61L 2/18

(57) ABSTRACT

A sterilization assembly and methods of use for sterilizing various instruments or surfaces are described herein. The assembly may generally comprise an elongate housing defining a storage lumen and an opening, a plurality of cartridges aligned adjacent to one another within the storage lumen where each cartridge has a cleaning element, a biasing mechanism configured to bias the plurality of cartridges towards the opening of the storage lumen, and a release mechanism positioned along the elongate housing and adjacent to the opening. Actuation of the release mechanism may release at least one cartridge at the opening for ejection. The assembly may also comprise an actuator which oscillates the cleaning element or the at least one cartridge relative to the elongate housing. Furthermore, a processor in electrical communication with the actuator which is programmed to monitor operation of the actuator and/or positioning of the plurality of cartridges may also be incorporated.

### CROSS-REFERENCE TO RELATED APPLICATIONS

This application claims the benefit of priority to U.S. Prov. Apps. 61/989,318 filed May 6, 2014 and 62/083,777 filed Nov. 24, 2014, each of which is incorporated herein by reference in its entirety.

### FIELD OF THE INVENTION

The present invention relates generally to sterilization assemblies and their methods of use. More particularly, the present invention relates to sterilization assemblies which provide ergonomic and inexpensive devices to facilitate the sterilization of various surfaces and instruments such as IV connection ports, dialysis access ports, medication vial septums, skin surfaces prior to percutaneous access, etc.

### BACKGROUND OF THE INVENTION

Hospital and CDC guidelines require cleaning of IV ports with propyl alcohol or chlorhexidine whenever accessed. The current widespread system requires the practitioner to obtain an alcohol pad, open the packaging, retrieve the saturated wipe, and scrub the area to be sterilized for at least fifteen seconds. Through surveys and observations, compliance with these recommendations is generally poor, especially among physicians. A significant percentage of practitioners do not clean IV ports before use and when they do, the time and technique to properly clean IV ports is inadequate. This practice seems archaic, poorly thought out, time consuming, without sufficient practitioner compliance and in grave need of improvement especially since noncompliance can contribute to hospital acquired infections that cost the healthcare system billions of dollars every year.

Other conventional cleaning methodologies have included the following the use of alcohol-saturated pads, e.g., Curos® caps (Ivera Medical Corp., San Diego, Calif.) which contain an antiseptic impregnated sponge that remains on female luer locks IV ports and sterilize in a minimum of three minutes. However, this is not practical in clinical situations requiring multiple injections in less than three minute intervals (e.g., in the operating room).

The Bard Site-Scrub® IPA (C.R. Bard Inc., Murray Hill, N.J.) cleaning device typically requires multiple steps to utilize and has been shown in studies to be an inferior sterilizer compared to alcohol pads. Hub filters are another cleaning device but these are not recommended by the CDC due to easy clogging and because such filters do not eliminate the need to sterilize the port with alcohol anyway. Finally, Hyprotek (Hyprotek, Inc., Spokane, Wash.) is a thorough cleaning solution above and beyond what the CDC recommends. However, this system requires too many complicated components and steps and is therefore generally not performed by practitioners.

Accordingly, there exists a need for methods and devices which are efficacious and safe in facilitating the disinfection of instruments such as IV ports.

### SUMMARY OF THE INVENTION

The cartridges contained within the cleaning device may be co-axially aligned or otherwise stacked upon one another such that the cartridges may seal and/or abut against an adjacent cartridge. Because the cleaning device is readily transportable for use or storage, the cleaning device may be attached to various surfaces or objects, such as an IV pole, to provide ready access to the practitioner for cleaning, sterilizing, or disinfecting any number of surfaces or instruments. The cleaning device allows for the efficient cleaning of instruments such as needleless IV ports, etc. without touching the injection port and holding only the syringe to avoid contamination. This system may also minimize handling of IV ports and other vulnerable areas of the IV system.

Generally, the cleaning assembly may comprise an elongate housing defining a storage lumen therein and an opening at a distal end of the storage lumen, one or more cartridges aligned adjacent to one another within the storage lumen with each cartridge having an alignment feature which translatably aligns the cartridges relative to the storage lumen, wherein each cartridge has a first surface and a second surface opposite to the first surface which has a cleaning element positioned upon and extending from the second surface such that the cleaning element from a proximal cartridge is positionable in proximity to the first surface of an adjacent distal cartridge. The assembly may further comprise a release mechanism positioned along the elongate housing and adjacent to the opening, wherein actuation of the release mechanism releases at least one cartridge positioned at the opening for ejection from the elongate housing. The assembly may also comprise an actuator within or along the elongate housing and in communication with the at least one cartridge positioned at the opening wherein activation of the actuator oscillates the cleaning element or the at least one cartridge relative to the elongate housing, and a processor in electrical communication with the actuator, wherein the processor is programmed to monitor operation of the actuator and/or positioning of the one or more cartridges.

In use, one method of disinfecting a region of interest with the cleaning assembly may generally comprise positioning a first cleaning element of a first cartridge in proximity to a distal opening of a storage lumen defined through an elongate housing, activating an actuator which moves the cleaning element or the first cartridge relative to the elongate housing to clean the region of interest when contacted by the cleaning element, actuating a release mechanism positioned along the elongate housing and adjacent to the opening such that the first cartridge is ejected from the elongate housing and a second cleaning element of a second cartridge is positioned within the opening, and monitoring an operation of the cleaning assembly via a processor in communication with the actuator, wherein the processor is programmed to monitor positioning of the one or more cartridges.

Another method of disinfecting a region of interest with a cleaning assembly may generally comprise positioning a first cleaning element of a first cartridge in proximity to a distal opening of a storage lumen defined through an elongate housing, removing a seal from the first cartridge such that the first cleaning element is exposed at the distal opening, activating an actuator which moves the first cleaning element or the first cartridge relative to the elongate housing to clean the region of interest when a pressure is imparted upon the first cleaning element, stopping the actuator after a predetermined period of time has lapsed, and ejecting the first cartridge from the distal when the pressure is removed from the first cleaning element and a second cleaning element of a second cartridge is positioned within the opening.

Yet another method of disinfecting a portion of an instrument may generally comprise attaching the instrument to be cleaned to a securing arm which is positioned in an initial configuration in proximity to a platform, actuating the securing arm to reposition the portion of the instrument to be cleaned from the initial configuration and into contact against a first cleaning element of a first cartridge positioned in proximity to a distal opening of a storage lumen defined through an elongate housing which is secured to the platform, activating an actuator which moves the cleaning element or the first cartridge relative to the elongate housing and the platform to clean the portion of the instrument, and repositioning the securing arm and the portion of the instrument back to the initial configuration after the portion of the instrument has been cleaned.

In yet another variation of the cleaning assembly, the assembly may generally comprise an elongate housing defining a storage lumen therein and an opening at a distal end of the storage lumen, one or more cartridges aligned adjacent to one another within the storage lumen with each cartridge having an alignment feature which translatably aligns the cartridges relative to the storage lumen, wherein each cartridge has a first surface and a second surface opposite to the first surface which has a cleaning element positioned upon and extending from the second surface such that the cleaning element from a proximal cartridge is positionable in proximity to the first surface of an adjacent distal cartridge, a release mechanism positioned along the elongate housing and adjacent to the opening, wherein actuation of the release mechanism releases at least one cartridge positioned at the opening for ejection from the elongate housing, an actuator within or along the elongate housing and in communication with the at least one cartridge positioned at the opening wherein activation of the actuator moves the cleaning element or the at least one cartridge relative to the elongate housing;

a pressure sensor in communication with at least one cartridge and the processor, wherein a pressure applied to the at least one cartridge is sufficient to activate the actuator to move the cleaning element, and a processor in electrical communication with the actuator, release mechanism, and pressure sensor.

The cartridges may generally comprise a retaining body having a wall which defines a receiving channel and an opening, at least one alignment feature which projects radially from an outer surface of the wall and which is configured to translatably align the retaining body, a liner positioned proximally upon a floor within the receiving channel, a cleaning element positioned within the receiving channel and distal to the liner, wherein the cleaning element defines a cavity sized for receiving an injection port within, and a seal which retains the cleaning element within the receiving channel and is removably attached around the opening of the retaining body.

The device may generally comprise a body which may optionally include a cover secured upon a platform. The body may hold a cartridge assembly, e.g., one or more cleaning cartridges which may be axially aligned such that cartridges are stacked upon one another. A distal cartridge may be positioned at a distal opening of the device which exposes a cleaning element for use and an advancement mechanism may be located near or in proximity to the distal opening for securing the cartridge for use as well as ejecting a spent cartridge and advancing another cartridge for subsequent use.

A release actuator, e.g., a button or mechanism, may be coupled to the advancement mechanism for initiating an ejection and advancement of a fresh cartridge. A cartridge actuation, e.g., spring or other biasing mechanism, may be positioned within a channel defined along the platform between a surface within the device and a pusher element to provide an actuation force pressing against the plurality of cartridges. The cartridge actuation may provide for a continuous force which presses against the plurality of cartridges to push the cartridges distally through the channel and out of the distal opening. The actuation mechanism provides a stop for controlling the securement, ejection, and advancement of the individual cartridges against the force provided by the cartridge actuation. The pusher element may provide a smooth and even interface between the cartridge actuation and the proximal terminal cartridge. A pusher interface located along a distal surface of the pusher element may define a projection or raised surface for contacting or abutting against the terminal cartridge.

The cartridge variation shown may each define a projection along a first side of the cartridge (described in further detail below) and may further define a guide or key (such as a groove or notch) along a second side of the cartridge opposite to the projection. This guide or key may interface and slide along a guiding projection or rail defined along the channel of platform to maintain the orientation of the cartridge assembly as well as to facilitate its movement while being translated through the channel.

Generally prior to each use, the practitioner may depress the release actuator to activate the advancement mechanism to discard a used cartridge positioned at the distal opening of the device or to unseal a cartridge positioned at the distal opening. If present, the used cartridge may be ejected to expose a fresh cleaning element on a subsequent cartridge which may be advanced distally by the cartridge actuation into a secured position within the distal opening for use, or an unused cartridge may have a seal removed for use. Optionally, the device may also incorporate a motor or actuator which may impart some additional motion or movement to the exposed cartridge or to the device to enhance cleaning by the cartridge upon the surface or instrument to be sterilized, as described in further detail herein. The motor or actuator may be activated manually or it may be activated automatically when the cleaning element has a predetermined pressure applied to it so that the amount of force and friction remains consistent in cleaning the surface or instrument. The additional imparted motion or movement may reduce the time needed to disinfect by increasing the friction imparted to clean off bacteria, biofilms, etc. Alternatively, additional cleaning modalities may also be incorporated to further enhance cleaning, e.g., ultrasound, infrared, ultraviolet, etc. The motor or actuator may be activated for a predetermined period of time.

Additional cartridges may be ejected and advanced within the device until the cleaning or disinfecting is completed or until all cartridges have been expended from the device. Alternatively, use of the device may be limited to a single cartridge. In either case, additional cartridges may be inserted or positioned within the device for further use, e.g., in stacks of 20 cartridges. In the variation where the motor or actuator may be activated automatically with the application of a predetermined pressure upon the cleaning element, once the pressure is released, e.g., when the device is removed from the surface or instrument being cleaned, the used cartridge may be automatically ejected and the next cartridge may be advanced ready for use or ready to be unsealed.

Many different variations of the cartridges may be utilized with the cleaning device. The cartridge may have various shapes in alternative variations, e.g., circular, elliptical, rectangular, square, etc. A cartridge lip may extend in an annular or partially circumferential projection extending radially from the cartridge body to form an annular shoulder and a receiving platform which forms a surface upon which a cleaning element may be attached to project distally. The cleaning element may be formed of an absorbent material such as foam, sponge, etc. which is able to retain a cleaning, disinfecting, or antiseptic fluid or agent within. The cleaning element may also comprise any number of open/semi-open/closed cell foams, split microfiber and other materials, etc. The cleaning element may also be formed of an ionically charged material such that the cleaning element is positively charged in order to attract bacteria away from the surface or instrument being cleaned as many pathogenic bacterial cell walls have a weak negative charge between pH 5-7.5. In other variations, the cleaning element may instead be negatively charged or it may optionally incorporate a magnet or electromagnet at the base of the cartridge to attract bacteria. Various combinations of a positive or negative charge as well as the use of a magnet or electromagnet may be utilized.

In an alternative variation, a receiving cavity may be formed to extend from the annular shoulder to facilitate positioning and retention of the cleaning element upon the cartridge. The cleaning element may still extend distally beyond the receiving cavity at least partially to allow for the cleaning element to clean flat surfaces.

A depression may be defined at least partially within the surface or receiving cavity to allow for the partial flexing of the cleaning element into the receiving cavity. This partial flexing may allow for the cleaning element to collapse around or at least partially envelope the outer surfaces of an instrument such an IV port for cleaning when the IV port is urged or pressed against the cleaning element and into the depression.

A cartridge receiving cavity may be defined to extend within the cartridge body to accommodate the cleaning element of an adjacently positioned cartridge. When stacked or aligned, the annular shoulder of one cartridge may abut against the bottom surface or cartridge lip of a second cartridge positioned distally. In this manner, each of the cartridges may be aligned and maintained in a collinear arrangement such that each adjacent cartridge is separated at a predetermined distance from one another. This arrangement also allows for each cartridge to function as a seal for the cleaning element of each cartridge positioned just proximal. The cartridge lip may also facilitate the retention or securement of the cartridge within the device. A projection may extend from a first side of the cartridge as a tab to facilitate the cartridge retention and/or securement while a guide or key (such as a groove or notch) may be defined along a second side of the cartridge opposite to the projection to maintain the orientation of the cartridge within the device as well as to facilitate the translation of the cartridge along a guide or rail within the device.

Another variation of the cartridge may have a single cartridge having a cartridge body and further having a cartridge lip located annularly or at least partially circumferentially around the cartridge body. The body may further define a receiving cavity for retaining a cleaning element within and an optional retaining and/or advancement cap may also be included for placement over or within the receiving cavity. The cap may define one or two tabs which may extend radially from the cap and the cartridge body may define corresponding tab receiving portions to accommodate the tabs such that the tabs extend through and beyond the receiving portions such that the tabs may be used to eject the cap from the cartridge either manually or automatically by the device during use.

Regardless of the cartridge configuration, each of the cartridges may be stacked upon one another or otherwise collinearly aligned to allow for the use and advancement of the cartridges. The number of cartridges when the device is fully loaded may vary anywhere from one or more cartridge but the cleaning device may generally hold between, e.g., 15 to 30 cartridges. While the cartridges may each function as a seal (e.g., compression seal) for an adjacent cartridge, one or more of the cartridges may include a retaining cap, e.g., for the terminal cartridge, in place of a cleaning element. Alternatively, cartridges having a retaining cap may be positioned in an alternating manner in between cartridges having cleaning elements.

Yet another variation of the cartridge may have a cup-shape configuration with a diameter of, e.g., 9 mm to 10 mm. In this variation, the cartridge may form a cup-shaped body with one or more guides defined along the outer surface of the body. The guides may be shaped to project from the surface and extend longitudinally to function as guide rails as the cartridges are translated through the cleaning device during use. The body may form a receiving cavity within which a cleaning element may be placed. An optional removable cap or cover may also be included. Moreover, the cleaning element may remain compressed within the cavity when the cap is in place.

The cleaning element itself may also be configured in various shapes or may be configured to interact with the various cartridge bodies in predetermined ways depending upon the type of surface or instrument to be cleaned. With the cleaning element configured into, e.g., a tubular shape, the cleaning element may be used to contact and clean any flat surface (including a skin surface). The cleaning element may also be used to clean an instrument, e.g., IV port, luer fitting, etc., which may be pressed into the cleaning element which may collapse or at least partially envelope the instrument for cleaning not only the distal surfaces of the instrument but also its side surfaces.

In another variation, the cleaning element may be positioned to rest upon an annular shoulder above a depression defined within the receiving cavity of cartridge body. The cleaning element may still be used to clean any flat surface but the addition of the depression may allow for the cleaning element to deform within the depression to collapse or at least partially envelope the instrument. The cleaning element may reside within the cartridge body and may also define a concave surface which may extend within the cleaning element to facilitate the cleaning of instruments or surfaces having projections or extending features.

Yet another variation is comprised of a central portion surrounded by an annular portion within the cartridge body. This variation may be suited to receive an instrument within the cleaning element by collapsing the central portion and allowing the sides of the instrument to become completely enveloped by the interior surfaces of the annular portion. The annular portion may define any number of shapes for receiving an instrument and the annular portion may also be configured to be fully circumferential or just partially circumferential with respect to the central portion.

In yet another variation, the cleaning element may be configured to have a central portion having a convex surface as well as an annular portion having a convex surface to collectively form the cleaning element. This configuration may not only allow the cleaning element to clean concave surfaces but may also allow for the cleaning of instruments which may be inserted within the cleaning element such that the annular portion may clean the sides of the instrument.

Regardless of the cleaning element configuration, any of the variations described may be used with any of the cartridge body variations also described. Moreover, while each of the cartridges within a cleaning device may be uniform, any of the various configurations of the cartridges as well as cartridges having any of the various configurations of the cleaning elements may be used in any number of combinations or any order of cartridges within a single cleaning device, if so desired.

Turning now to the advancement mechanism for securing, ejecting, and advancing the cartridges within the cleaning device, a cleaning device may be comprised of a body and a channel defined therethrough for holding the one or more cartridges. Generally, the advancement mechanism may be located near or at the distal end of the cleaning device in proximity to the distal opening through which the cartridges are positioned for use and ejected from.

One variation of an advancement and retaining mechanism which may be used in the cleaning device utilizes a circularly shaped ring-like retaining member which has at least one gap or split positioned in proximity to the advancement mechanism. An optional gap or split may also be defined on the opposite side of the retaining member as well. When in its secured configuration, the retaining member may be urged or biased to towards one another such that the retaining member may close upon the retaining lip defined circumferentially around the terminally positioned cartridge to allow for the terminal cartridge to be used for cleaning. Pressing upon the advancement mechanism may force the retaining member away from one another allowing for the retaining lip to advance distally past the retaining member such that the terminal cartridge is ejected.

A controller and/or processor may be incorporated into the cleaning device for providing various controls or monitoring features as well as a motor or actuator in proximity to the cartridges (as described in further detail herein).

Another variation may use at least two retaining arm members positioned on opposite sides of the terminal cartridge such that each arm member defines a retaining shoulders extending transversely and towards the retaining lip of the first cartridge body. Each arm member may also define an angled inner surface which is tapered away from the retaining lip. Proximal to the retaining arm members, corresponding release arm members may be positioned between the retaining lip of the first cartridge body and the retaining aim members. Each of the release arm members may define an angled outer surface.

Once the advancement mechanism is depressed, each of the release arm members may be urged distally through the cleaning device and towards the retaining arm members. As the angled outer surfaces of the release arm members contact against the angled inner surfaces of retaining arm members, the retaining arm members may be forced radially outward away from the first cartridge such that the retaining lip becomes disengaged from the retaining shoulders. This disengagement allows for the first cartridge to be ejected through the distal opening and for the second cartridge to be advanced distally into position within the distal opening. As the retaining arm members are forced radially outward, the release arm members are simultaneously forced radially inward against the body of the second cartridge and against the second cartridge lip to prevent the second cartridge from also being ejected. Retraction of the release arm members may accordingly allow for the retaining arm members to reposition themselves into their initial securement position where the retaining shoulders may now rest against the second cartridge lip to secure its cleaning element within the distal opening of the cleaning device for use. This process may be repeated as desired or needed until the cartridges are exhausted from the cleaning device.

In another variation, rather than having the release arm members positioned on opposite sides of the cartridges, the release arm members may instead be positioned on a single side of the cartridges such that the cartridge engagement regions are positioned on opposite sides of a projection extending from the cartridge body.

Additional variations for release arm members and advancement mechanisms may include the release arm members positioned along a single side of the cartridge adjacent to the advancement mechanism. When the advancement mechanism is depressed, a tapered pushing member having tapered interfaces defined along both sides and extending from the mechanism may force the release arm members apart from one another. As the arm members draw apart the cartridge may be released to advance distally for ejection from the cleaning device.

Another variation of an advancement mechanism may have a cartridge engagement region sized to engage the retaining lip of the second cartridge while pushing distally against the retaining lip of the first cartridge. The mechanism may define an angled surface located along the distal edge of the mechanism to push distally against the retaining lip to eject the first cartridge from the cleaning device. Once the first cartridge has been ejected, the mechanism may be disengaged from the retaining lip allowing for the distal advancement of the second cartridge. In another variation, a distal portion of the mechanism may be attached to a coupling member via a pivot and the coupling member may extend to form a release arm member. A corresponding release arm members may be located on an opposite side of the cartridges. The release arm member itself may be restrained via a pivot along its length. As the mechanism is depressed, the coupling member may force the release arm member to move radially away from the cartridges via the pivot such that a proximal engagement surface of the release arm member and a corresponding proximal engagement surface of the release arm member are angled away from the cartridges to release the retaining shoulder of first cartridge. Once the mechanism is released, the second cartridge may be unconstrained by the proximal shoulder to then advance distally until the shoulder abuts the proximal engagement surfaces of the release arm members.

In yet another variation, the advancement mechanism may be restrained via a pivot located at its distal end of the mechanism. The mechanism may also include a retaining member extending transversely from the pivot and an angled surface defined to extend proximally from the pivot. In its engaged state, the retaining lip of the first cartridge may be secured between the retaining member and angled surface. However, when the mechanism is actuated, the mechanism may rotate about pivot such that the angled surface may push distally against the retaining lip while simultaneously disengaging the retaining member to allow for the retaining lip to advance distally.

Yet another variation includes release arm members each having retaining members which extend transversely and radially inward as well as angled members which also extend radially inward at an angle. The release arm members may be attached via pivots such that when the release mechanism is depressed, the release arm members may pivot about each respective pivot such that the angled members angle inwardly against the first cartridge and the retaining members angle outwardly away from the first cartridge.

Regardless of the variation of the release mechanism, any of these variations described is fully and expressly intended to be utilized with any of the cartridge variations described herein and in any number of combinations, as practicable, and furthermore is not limited to any particular combination of features.

In any of the variations described, a motor or actuator in communication with at least the terminal cartridge may be optionally incorporated for providing motion to the cleaning element. This motion may enhance the cleaning action of the cartridge and cleaning element when contacted against the surface and/or instrument to be cleaned. A controller and/or processor may also be incorporated into the cleaning device for providing various controls or monitoring various features as well as controlling the motor or actuator.

The controller and/or processor, for instance, may be programmed to track usage time, location of usage, the power used for the cleaning device as well as other parameters such as the time spent on cleaning particular instruments. The controller and/or processor may also be programmed to track which patients the cleaning device has been assigned to and/or which practitioner has utilized the device as well as parameters such as the physical location of the cleaning device. Additionally, the cleaning device may further include memory capacity as well as wireless transmission and reception capabilities. Hence, the controller and/or processor may be electrically coupled to on-board memory for storing various parameters (e.g., for downloading) or for wirelessly transmitting these parameters to a remote location such as a server through various methods, e.g., Bluetooth®, radio signals, infrared, etc. where the data may be viewed or processed by another party remotely, e.g., through access over an internet based cloud storage system. Additionally, the controller and/or processor may also be programmed to time the cleaning of an instrument. For instance, the controller and/or processor may be programmed to actuate the cleaning element for a minimum period of time, e.g., 1 to 15 seconds or more, once an instrument has started cleaning to provide for sufficient disinfection. The period of time may be preset to any length of time.

The actuator may comprise a motor in electrical communication with the controller and/or processor. A mass element may be eccentrically coupled to the actuator such that activation of the actuator may rotate the mass element which imparts a vibrational motion or pulses to the actuator and to a vibrational conductor. The vibrational motion may be transmitted via the vibrational conductor which may comprise any vibrationally conductive material such as a metal or plastic to conduct the vibrations into contact against the terminally positioned cartridge positioned at the distal opening. The vibrational conductor may thereby conduct the vibrations such that the cleaning element positioned at the distal opening may vibrate or move accordingly to enhance the cleaning action.

In order to input various parameters into the cleaning device, variations of the cleaning device may optionally communication through various methods such as RFID, QR codes, two-dimensional bar codes, near field communication, infrared, etc. The cleaning devices may also be optionally stored in a charging or docking station which may provide for charging of power. The station may also include an electronics unit which may be in communication, either wireless or wired, with the cleaning device. Various information or parameters stored by the cleaning device may be downloaded to the docking station which may also be connected via a wireless or wired connection to a remote serve such as a central electronic server system used in a hospital setting.

Another variation of a cleaning device may incorporate a magazine having an elongate magazine housing which holds one or more cartridges aligned within. The cartridges may have a cartridge actuation mechanism, e.g., spring or other biasing member, which may push the cartridges distally through the magazine housing. A proximal portion of the magazine housing may also be comprised of a vibrationally conductive material. The entire magazine may be removably inserted within the body of the cleaning device as an assembly such that the distal opening of the magazine may align with the distal opening of the cleaning device such that the terminal cartridge is positioned within the distal opening for use. The cleaning device may have an advancement mechanism which may also align and couple with the cartridges held within the magazine. The cleaning device may also have an actuation assembly, such as a motor, controller and electronics housing, and power supply within the cleaning device such that the actuation assembly contacts the proximal portion of the magazine for optionally transmitting vibrations or motion to the terminally positioned cartridge.

Regardless of the actuation configuration, any of the variations described may be used with any of the cartridge body variations and advancement mechanisms described, as practicable. Hence, the variations shown are not intended to be limiting but may encompass any combination of features between the various mechanisms and features.

Any of the cartridge variations described may be sealed via an adjacent cartridge. However, any of the cartridge variations may also optionally incorporate an individual covering or seal to maintain sterilization of the cleaning element prior to its use. One variation of a flexible covering may have a first tab and a second tab which are each positioned to extend tangentially in opposite directions. The flexible covering may be fabricated from various materials, e.g., peelable metal foils, plastic films, wax paper, etc. A tear point (e.g., a weakened portion of the tab) may be optionally defined to extend diametrically across the covering and perpendicularly relative to the tabs so that when the tabs are engaged and pulled (e.g., manually or otherwise) in opposing directions, the covering may split along the tear point to expose the cleaning element contained within the cartridge for use.

Yet another variation of a covering may have a single tab which defines an opening at its distal end and has an attachment to the covering at a proximal end of the tab. Prior to use, the tab may be folded over upon itself such that it extends back over the covering such that the opening of the tab extends over the cartridge while the covering remains sealed to the cartridge.

In addition to the various combinations of features, the cleaning device itself may be optionally configured with one or more attachments to facilitate its use. One example of a cleaning device may optionally include an attachable retractable cable housing. Another attachment option may include a fluid supply attachment which may be attached to the cleaning device. Another attachment option may also include a fan or blower which may be removably attached to the cleaning device. The fan or blower may be used to speed up the drying of the cleaned surface or instrument. Any of these attachments may be optionally incorporated with any of the device features and mechanisms described herein, as so desired.

Because any of the cleaning device variations described and any of their combinations are portable and facilitate their use, the cleaning device may be used manually by the practitioner. Alternatively, use of the cleaning device may be automated to some degree.

Furthermore, an optional data log may also be incorporated with the cleaning device. During use of the cleaning device, the controller and/or processor may be programmed to log information such as date and time for each use. This information may be downloaded to a server system where it may be compiled and compared to the overall case statistics of a setting, e.g., hospital, to ensure that the cleaning device was used each time an instrument, such as a port, was accessed. The data log could be expanded and integrated deeper into the hospital system by logging location, user, and or patient and this tracking of information could increase accountability and better enable outcome tracking.

The cleaning device has tracking abilities, as described herein, where it can send the various recorded parameters to a particular electronic health record. The electronic tracking system can automatically determine if there are a series of mismatches between medication administration and device use which can be reported electronically back to the clinician or to an infection control professional. Another way the tracking system can be used is by finding an infection and then looking for the mismatch between medication administration and device use and thereby the offending clinician.

These cleaning systems are intended to be utilized with any of the cleaning device variations described herein and in any combination.

### BRIEF DESCRIPTION OF THE DRAWINGS

FIGS. 1A and 1B illustrate perspective views of one variation of a cleaning device containing one or more cleaning cartridges

FIG. 1C illustrates a perspective view of the cleaning device with the plurality of cleaning cartridges removed.

FIG. 2A illustrates a perspective view of one variation of a cleaning cartridge.

FIGS. 2B and 2C illustrate cross-sectional side views of variations of the cleaning cartridge.

FIGS. 2D and 2E illustrate cross-sectional side views of other variations of the cleaning cartridge where the cleaning element may contain a charge and/or where the cartridge may incorporate a magnet or electromagnet.

FIGS. 3A and 3B illustrate perspective views of another variation of a cartridge having a removable cap with tabs which may overhang the cartridge to provide a surface or mechanism for expelling the cartridge and/or cap.

FIGS. 3C to 3E illustrate side views of the cartridge with the cap positioned upon the cartridge and also removed from the cartridge.

FIG. 4A illustrates a cross-sectional side view of a plurality of stacked cartridges.

FIG. 4B illustrates a cross-sectional side view of stacked cartridges having at least one of the cartridges fitted with a cap.

FIG. 5A illustrates a cross-sectional side view of stacked cartridges sealed upon one another with an intermediate cap.

FIG. 5B illustrates a cross-sectional side view of stacked cartridges sealed upon one another.

FIGS. 6A and 6B illustrate perspective views of another variation of a cartridge having guides along the body.

FIG. 6C illustrates an exploded perspective view of the cartridge of FIGS. 6A and 6B.

FIGS. 7A and 7B illustrate cross-sectional side views of examples of a cartridge cleaning a flat surface or a luer fitting.

FIGS. 8A and 8B illustrate cross-sectional side views of another variation of a cartridge having a defined relief area.

FIGS. 9A and 9B illustrate cross-sectional side views of a cartridge having a concave and convex surface, respectively, defined upon the cleaning element.

FIGS. 10A and 10B illustrate cross-sectional side views of another variation of a cleaning element having a central cleaning portion separate from an annular portion to facilitate cleaning of a port.

FIGS. 11A and 11B illustrate cross-sectional side views of another variation of a cleaning having a central portion and further defining a convex surface.

FIG. 12A illustrates a partial cross-sectional side view of the cartridges aligned adjacent to one another within the body of a cleaning device with an advancement mechanism positioned in proximity to the distal opening of the device.

FIG. 12B illustrates a perspective view of one variation of the body of the cleaning device having one or more rails or guides aligned longitudinally to facilitate the guidance and advancement of the cartridges.

FIGS. 13A and 13B illustrate perspective views of one variation of an advancement mechanism for retaining, advancing, and ejecting cartridges.

FIGS. 14A to 14C illustrate cross-sectional side views of another variation of an advancement mechanism.

FIG. 15 illustrates a cross-sectional side view of yet another variation of an advancement mechanism.

FIG. 16 illustrates a cross-sectional side view of yet another variation of an advancement mechanism.

FIGS. 17A to 17C illustrate end views of release mechanism variations for the advancement mechanism.

FIG. 18 illustrates a partial cross-sectional side view of yet another variation of an advancement mechanism.

FIG. 19 illustrates a partial cross-sectional side view of yet another variation of an advancement mechanism.

FIG. 20 illustrates a partial cross-sectional side view of yet another variation of an advancement mechanism.

FIG. 21 illustrates a partial cross-sectional side view of yet another variation of an advancement mechanism.

FIG. 22 illustrates a partial cross-sectional side view of yet another variation of an advancement mechanism.

FIGS. 23A and 23B illustrate perspective and side views of another variation of the cleaning device having an actuation motor positioned within the cleaning assembly.

FIG. 23C illustrates a perspective view of one variation of the actuation motor.

FIG. 23D illustrates an example of a cleaning device coupled either wirelessly or wired to a docking station.

FIGS. 24A and 24B illustrate perspective and side views of another variation of the actuation motor.

FIGS. 25A and 25B illustrate cross-sectional side views of a variation of the cleaning device having a magazine preloaded with one or more cartridges which may be placed into contact with an actuation motor.

FIGS. 25C and 25D illustrate cross-sectional side views of another variation of the cleaning device where the one or more cartridges may be aligned in a side-to-side manner.

FIGS. 25E to 25K illustrate another variation which may also utilize a magazine which contains one or more of the cartridges.

FIG. 26A illustrates schematically an example for enhanced cleaning actuation.

FIGS. 26B to 26E illustrate schematic variations for alternative enhanced cleaning actuation.

FIG. 27 illustrates a partial cross-sectional side view of an actuation motor mounted to a coupling head which may rotate a cartridge eccentrically.

FIG. 28A and 28B illustrate end and side views of an actuation mechanism for enhanced cleaning where the actuator is positioned adjacent to the stack of discs or cups and eccentrically coupled to a coupling head which partially rotates the terminal cartridge upon actuation.

FIG. 28C illustrates an end view of another variation of an actuation mechanism similar to the variation shown in FIG. 28A.

FIG. 28D illustrates an end view of yet another variation of an actuation mechanism where the motor is coupled to the coupling head via a rack-and-pinion type coupling.

FIG. 28E illustrates an end view of yet another variation of an actuation mechanism where the motor is eccentrically coupled to the coupling head via an off-center linkage arm.

FIGS. 29A to 29C illustrate top views of variations of a removable cap or cover which may be applied to any of the cartridge variations described herein where the cap or cover may be attached around a periphery of the cartridge with tabs extending tangentially and in opposite directions.

FIG. 29D illustrates a top view of another variation of a removable cap or cover having tabs extending from a periphery of the cap or cover in diametric opposite directions.

FIG. 29E illustrates a top view of another variation of a removable cap or cover having tabs extending in opposite directions and attached at either end of each tab adjacent to one another.

FIGS. 30A to 30C illustrate respective top and side views of another variation of a removable cap or cover having a tab extending radially from a first side of the cap or cover and which is folded or looped upon itself to extend from a second side opposite to the first side such that tensioning of the tab urges the cap or cover to peel from the cartridge in an opposite direction from the tab projection on the first side.

FIG. 31 illustrates a perspective view of one variation for positioning the tab and cap or cover of FIG. 30C for automated removal from a respective cartridge.

FIGS. 32A to 32E illustrate side views of the variation of FIG. 31 to show one variation for the automated removal of the cap or cover.

FIGS. 33A and 33B illustrate an assembly and perspective view of another variation of the device optionally incorporating a retractable cable and cable housing for facilitating manipulation and positioning of the housing dispenser while remaining attached to a platform such as an IV pole.

FIG. 33C illustrates an assembly view of yet another variation of the device retractably coupled to a cable housing which is separate from the cleaning device.

FIG. 34 illustrates a side view of another variation of a cleaning device optionally incorporating an atomizer

FIGS. 35A and 35B illustrate assembly views of another variation of a device which may be secured, removably or permanently, upon a platform attached, e.g., to an IV pole, where a hinged or pivoting arm may be configured to allow for the automatic positioning relative to the device and disinfecting of an instrument such as an IV port.

FIGS. 36A to 36F illustrate an example of the different steps for temporarily securing an instrument, such as an IV port, to an articulatable arm member which may be repositioned to bring the IV port into contact with the device for disinfection.

FIG. 37 illustrates a flow diagram showing how data logged from the cleaning device may be compiled for examination.

FIG. 38 illustrates a perspective assembly view of a device variation which may be assembled in a modular configuration with variable components.

### DETAILED DESCRIPTION OF THE INVENTION

The cleaning device and its variations described herein may generally be used to improve cleaning compliance and to sufficiently remove bacteria, biofilms, or other agents from surfaces and/or instruments such as IV ports. Generally, the cleaning device may be sized to be held in a single hand such that the device is pen-like which may be used in various environments or locations, e.g., operating room, endoscopy labs, catheter labs, emergency rooms, etc. Within the device, one or more cartridges may be stacked or aligned relative to one another where each of the one or more cartridges may be topped or incorporate a cleaning element such as a sponge. (As used herein, “cartridge”, “disc”, or “cup” may be used interchangeably.) This cleaning element may incorporate or be saturated with a cleaning agent (e.g., water, saline, detergent, etc.) or sterilizing solution (e.g., isopropyl alcohol, chlorhexidine, hydrogen peroxide or silver, or other antiseptic solution or combinations thereof, etc.).

The cartridges contained within the cleaning device may be co-axially aligned or otherwise stacked upon one another such that the cartridges may seal and/or abut against an adjacent cartridge. Because the cleaning device is readily transportable for use or storage, the cleaning device may be attached to various surfaces or objects, such as an IV pole, to provide ready access to the practitioner for cleaning, sterilizing, or disinfecting any number of surfaces or instruments.

The cleaning device allows for the efficient cleaning of instruments such as needleless IV ports, inject syringes, etc. without touching and holding only the syringe to avoid contamination. This system may also minimize handling of IV ports and other vulnerable areas of the IV system.

Cleaning Device

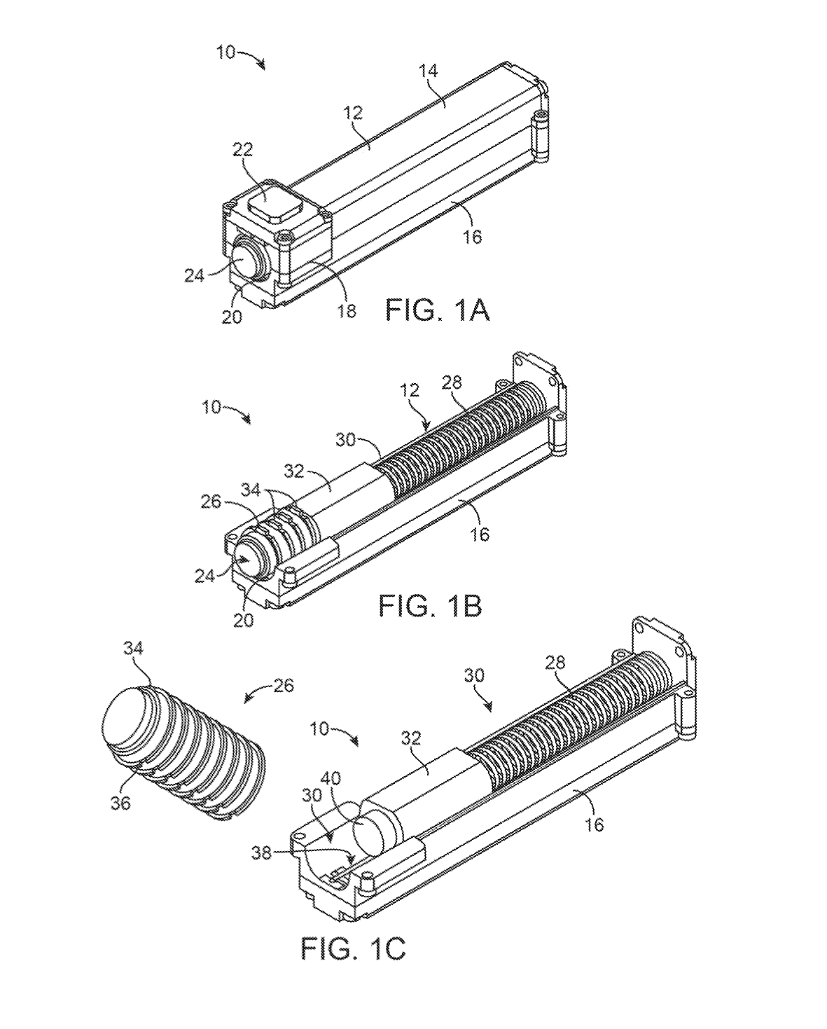

FIGS. 1A to 1C show one illustrative variation of the cleaning device 10 which may be sized suitably to be held in a single hand of the practitioner. The device 10 may generally comprise a body 12 which may optionally include a cover 14 secured upon a platform 16. The body 12 may hold a cartridge assembly 26, e.g., one or more cleaning cartridges 24 which may be axially aligned such that cartridges 24 are stacked upon one another. A distal cartridge 24 may be positioned at a distal opening 20 of the device 10 which exposes a cleaning element for use and an advancement mechanism 18 may be located near or in proximity to the distal opening 20 for securing the cartridge 24 for use as well as ejecting a spent cartridge 24 and advancing another cartridge 24 for subsequent use.

A release actuator 22, e.g., a button or mechanism, may be coupled to the advancement mechanism 18 for initiating an ejection and advancement of a fresh cartridge. FIG. 1B shows the device 10 with the cover 14 removed for illustrative purposes to show how a cartridge actuation 28, e.g., spring or other biasing mechanism, may be positioned within a channel 30 defined along the platform 16 between a surface within the device 10 and a pusher element 32 to provide an actuation force pressing against the plurality of cartridges 26. The cartridge actuation 28 may provide for a continuous force which presses against the plurality of cartridges 26 to push the cartridges distally through the channel 30 and out of the distal opening 20. The actuation mechanism 18 provides a stop for controlling the securement, ejection, and advancement of the individual cartridges against the force provided by the cartridge actuation 28. The pusher element 32 may provide a smooth and even interface between the cartridge actuation 28 and the proximal terminal cartridge. A pusher interface 40 located along a distal surface of the pusher element 32 may define a projection or raised surface for contacting or abutting against the terminal cartridge, as shown in FIG. 1C which illustrates the cartridges 26 removed from the device 10 for illustrative purposes.

The cartridge variation shown may each define a projection 34 along a first side of the cartridge (described in further detail below) and may further define a guide or key 36 (such as a groove or notch) along a second side of the cartridge opposite to the projection 34. This guide or key 36 may interface and slide along a guiding projection or rail 38 defined along the channel 30 of platform 16 to maintain the orientation of the cartridge assembly 26 as well as to facilitate its movement while being translated through the channel 30.

Generally prior to each use, the practitioner may depress the release actuator 22 to activate the advancement mechanism 18 to discard a used cartridge positioned at the distal opening 20 of the device 10. The used cartridge may be ejected to expose a fresh cleaning element on a subsequent cartridge which may be advanced distally by the cartridge actuation 28 into a secured position within the distal opening 20 for use. Optionally, the device 10 may also incorporate a motor or actuator which may impart some additional motion or movement to the exposed cartridge or to the device 10 to enhance cleaning by the cartridge upon the surface or instrument to be sterilized, as described in further detail herein. The additional imparted motion or movement may reduce the time needed to disinfect by increasing the friction imparted to clean off bacteria, biofilms, etc. Alternatively, additional cleaning modalities may also be incorporated to further enhance cleaning, e.g., ultrasound, infrared, ultraviolet, etc.

Additional cartridges may be ejected and advanced within the device 10 until the cleaning or disinfecting is completed or until all cartridges have been expended from the device 10. Alternatively, use of the device 10 may be limited to a single cartridge. In either case, additional cartridges may be inserted or positioned within the device 10 for further use, e.g., in stacks of 20 cartridges.

Cartridges

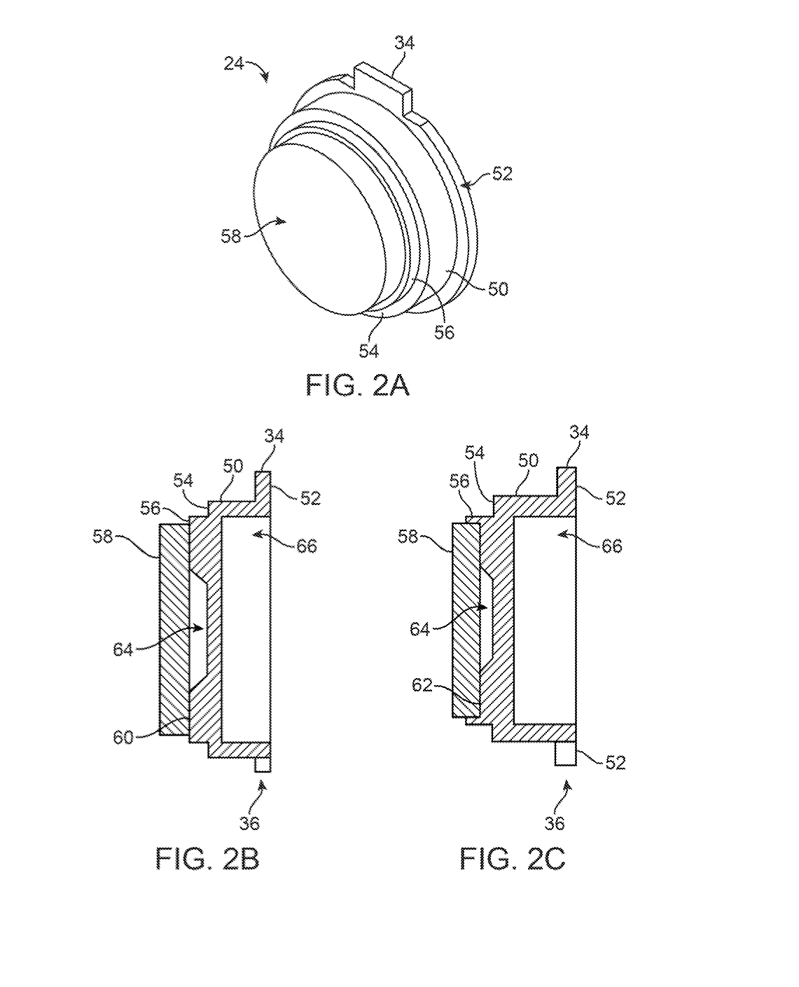

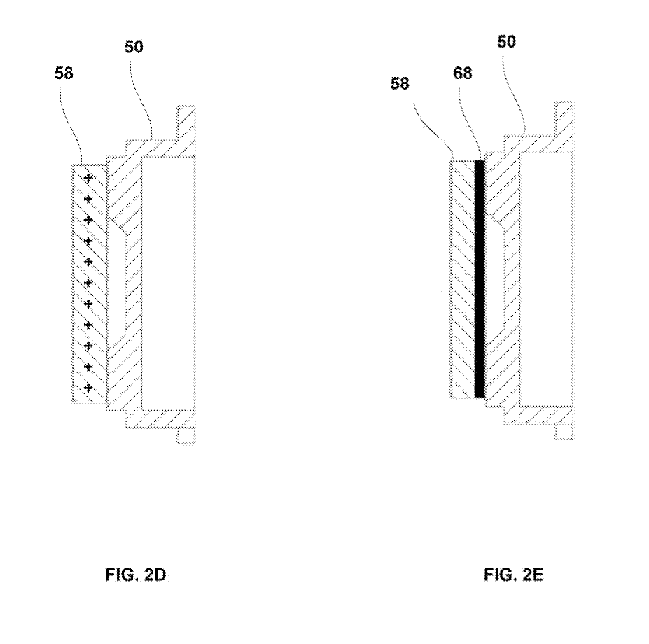

Many different variations of the cartridges may be utilized with the cleaning device 10. FIGS. 2A and 2B illustrate a perspective and cross-sectional side view of one variation of the cartridge 24 as having a cartridge body 50 which may be circular in shape. The cartridge 24 may have other shapes in alternative variations, e.g., elliptical, rectangular, square, etc. A cartridge lip 52 may extend in an annular or partially circumferential projection extending radially from the cartridge body 50 to form an annular shoulder 54 and a receiving platform 56 which forms a surface 60 upon which a cleaning element 58 may be attached to project distally. The cleaning element 58 may be formed of an absorbent material such as foam, sponge, etc. which is able to retain a cleaning, disinfecting, or antiseptic fluid or agent within. The cleaning element may also comprise any number of open, semi-open or semi-closed cell foams of different densities, split microfiber and other materials, etc. The cleaning element may also be formed of an ionically charged material such that the cleaning element is positively charged, as shown in FIG. 2D, in order to attract bacteria away from the surface or instrument being cleaned as many pathogenic bacterial cell walls have a weak negative charge between pH 5-7.5. In other variations, the cleaning element may instead be negatively charged or it may optionally incorporate a magnet or electromagnet 68 at the base of the cartridge to attract bacteria, as shown in FIG. 2E. Various combinations of a positive or negative charge as well as the use of a magnet or electromagnet may be utilized

In an alternative variation, a receiving cavity 62 may be formed to extend from the annular shoulder 54 to facilitate positioning and retention of the cleaning element 58 upon the cartridge 24, as shown in the cross-sectional side view of FIG. 2C. The cleaning element 58 may still extend distally beyond the receiving cavity 62 at least partially to allow for the cleaning element 58 to clean flat surfaces.

A depression 64 may be defined at least partially within the surface 60 or receiving cavity 62 to allow for the partial flexing of the cleaning element 58 into the receiving cavity 62. This partial flexing may allow for the cleaning element 58 to collapse around or at least partially envelope the outer surfaces of an instrument such an IV port for cleaning when the IV port is urged or pressed against the cleaning element 58 and into the depression 64.

A cartridge receiving cavity 66 may be defined to extend within the cartridge body 50 to accommodate the cleaning element 58 of an adjacently positioned cartridge. When stacked or aligned, the annular shoulder 54 of one cartridge may abut against the bottom surface or cartridge lip 52 of a second cartridge positioned distally. In this manner, each of the cartridges may be aligned and maintained in a collinear arrangement such that each adjacent cartridge is separated at a predetermined distance from one another. This arrangement also allows for each cartridge to function as a seal for the cleaning element 58 of each cartridge positioned just proximal. The cartridge lip 52 may also facilitate the retention or securement of the cartridge within the device 10. A projection 34 may extend from a first side of the cartridge as a tab to facilitate the cartridge retention and/or securement while a guide or key 36 (such as a groove or notch) may be defined along a second side of the cartridge opposite to the projection 34 to maintain the orientation of the cartridge within the device 10 as well as to facilitate the translation of the cartridge along a guide or rail within the device 10.

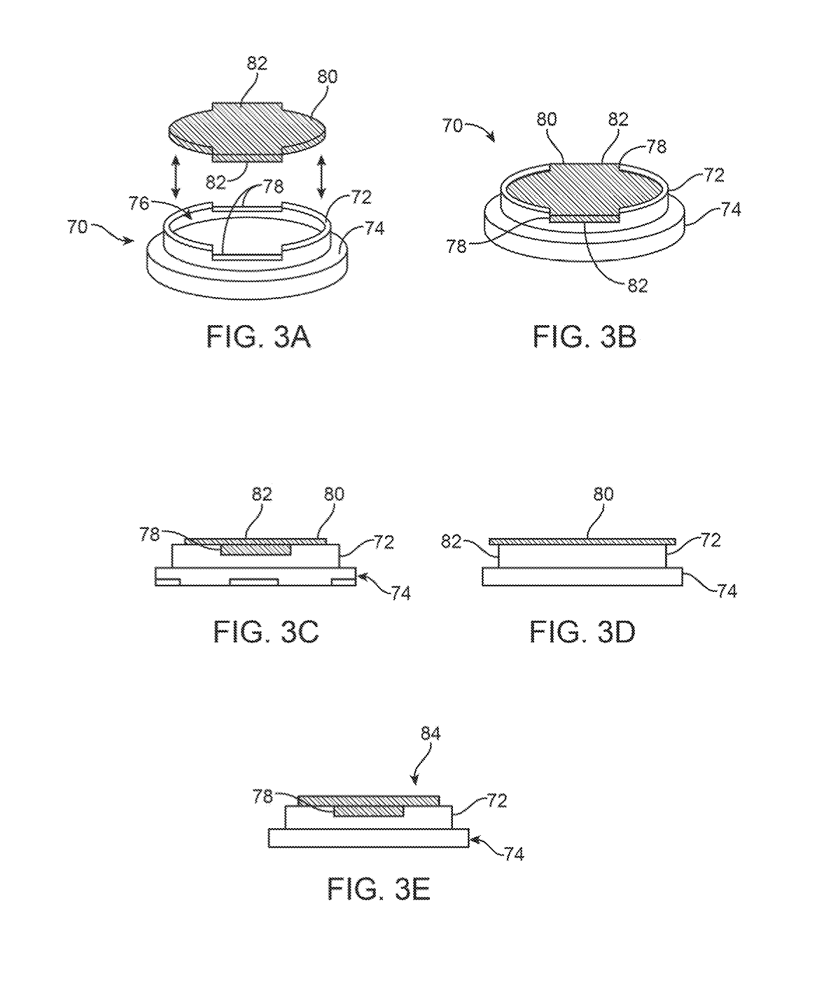

Another variation of the cartridge is shown in the perspective views of FIGS. 3A and 3B which illustrate a single cartridge 70 having a cartridge body 72 and further having a cartridge lip 74 located annularly or at least partially circumferentially around the cartridge body 72. The body 72 may further define a receiving cavity 76 for retaining a cleaning element 84 within and an optional retaining and/or advancement cap 80 may also be included for placement over or within the receiving cavity 76. The cap 80 may define one or two tabs 82 which may extend radially from the cap 80 and the cartridge body 72 may define corresponding tab receiving portions 78 to accommodate the tabs 82 such that the tabs 82 extend through and beyond the receiving portions 78 such that the tabs 82 may be used to eject the cap 80 from the cartridge either manually or automatically by the device during use.

FIGS. 3C and 3D illustrate side views of the cap 80 positioned upon the cartridge and illustrate how the tabs 82 may overhang from the cartridge body 72. When the cap 80 is placed upon the cartridge, the cap 80 may be retained through various mechanisms. For instance, the cap 80 may be held by interference between the tabs 82 and body 72 or they may be held by an adhesive holding the two components to one another. When the cap 80 is placed upon the cartridge, the cleaning element 84 may be compressed within the cartridge body 72 such that removal of the cap 80 may allow for the cleaning element 84 to decompress, as shown in the side view of FIG. 3E.

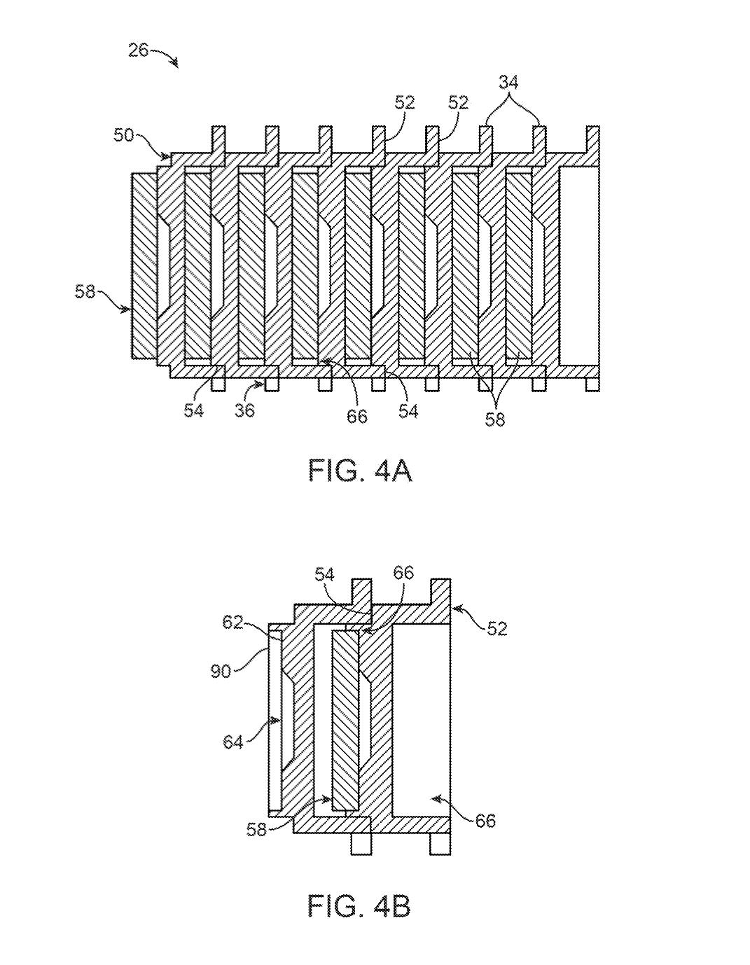

Regardless of the cartridge configuration, each of the cartridges may be stacked upon one another or otherwise collinearly aligned to allow for the use and advancement of the cartridges. FIG. 4A illustrates a cross-sectional side view of one variation of multiple cartridges which may be positioned within a cleaning device. The number of cartridges when the device 10 is fully loaded may vary anywhere from one or more cartridge but the cleaning device 10 may generally hold between, e.g., 15 to 30 cartridges. The example shown illustrates how the cleaning element 58 of a proximally positioned cartridge may be positioned correspondingly within the cartridge receiving cavity 66 of an adjacent distal cartridge while the lip 52 of the distal cartridge rests against the shoulder 54 of the proximal cartridge to maintain a preset distance between the adjacent cartridges to prevent damage to the cleaning element 58. While the cartridges may each function as a seal (e.g., compression seal) for an adjacent cartridge, one or more of the cartridges may include a retaining cap 90, e.g., for the terminal cartridge, in place of a cleaning element 58, as shown in the cross-sectional view of FIG. 4B. Alternatively, cartridges having a retaining cap 90 may be positioned in an alternating manner in between cartridges having cleaning elements 58.

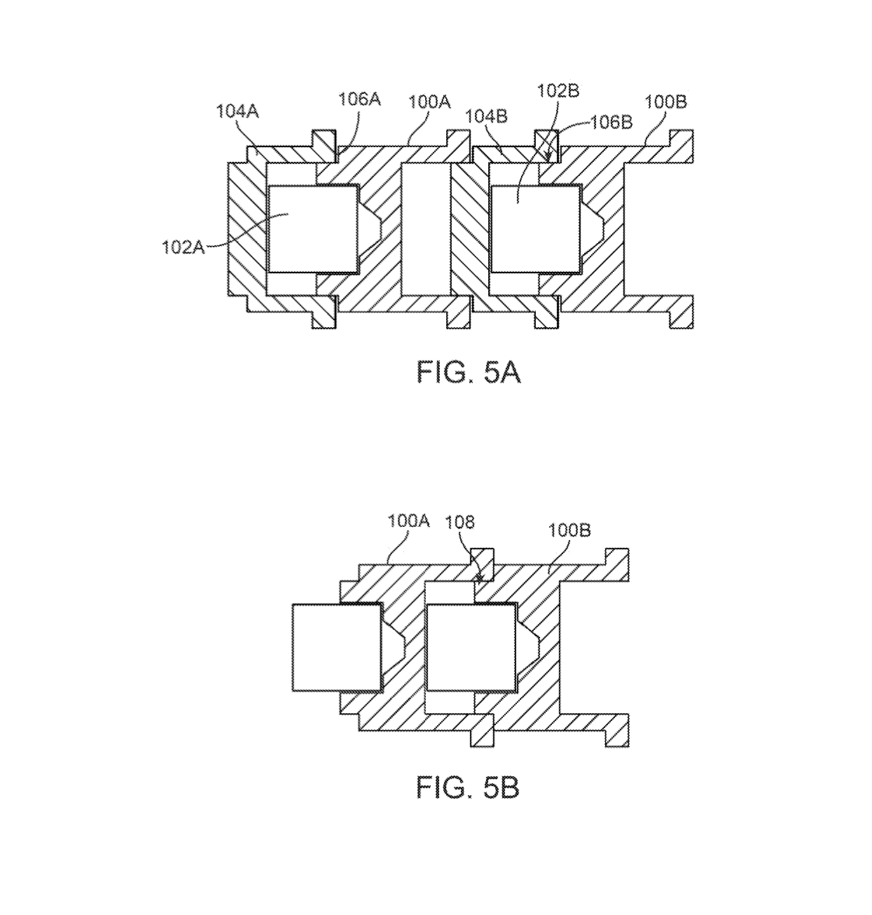

FIG. 5A illustrates a cross-sectional side view of another variation of the cartridges where cartridges configured as caps may be used in an alternating manner to seal the proximal cleaning elements. A first cartridge 100A having a first cleaning element 102A may be seen having a first cap 104A aligned and stacked distally to form a first cap-to-cartridge seal 106A. A second cap 104B may be aligned proximal to the first cartridge 100A to form a second cap-to-cartridge seal 106B. The second cap 104B may similarly cap the second cleaning element 102B of second cartridge 100B. The alternating cartridge and cap arrangement may be repeated for each of the cartridges. FIG. 5B illustrates a cross-sectional side view of another variation which may utilize the cartridges themselves to function as caps for a proximally adjacent cleaning element. In this variation, the cartridges 100A, 100B may each form an optional shoulder to facilitate the cartridge-to-cartridge seal 108 in order to keep the cleaning element sealed prior to use.

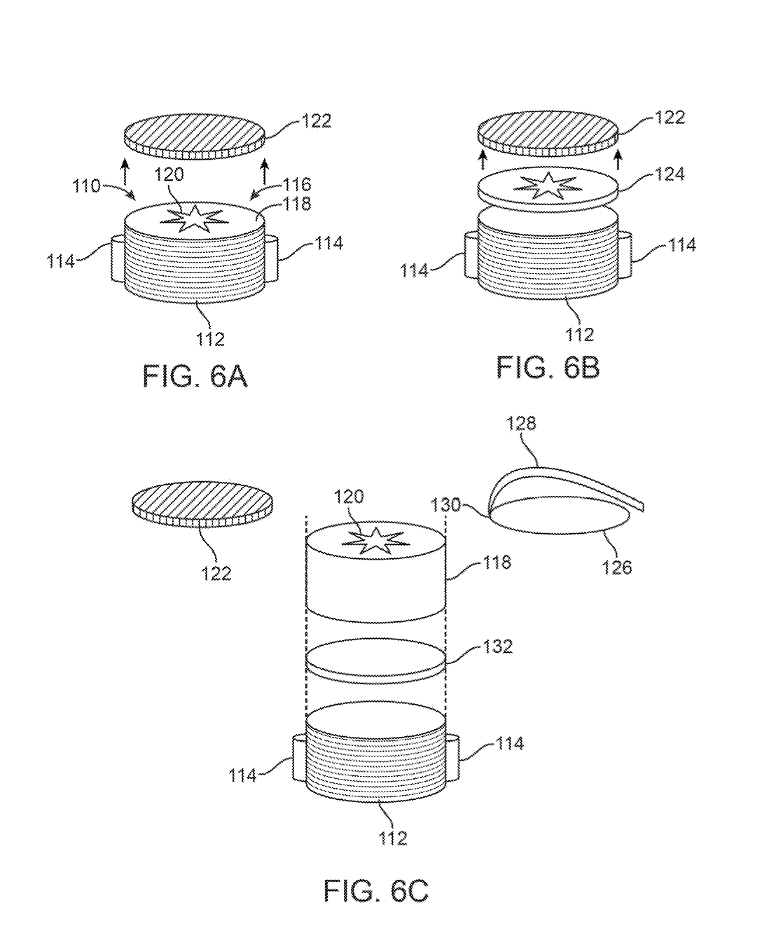

FIGS. 6A to 6C illustrate perspective views of yet another variation of the cartridge 110 having a cup-shape configuration with a diameter of, e.g., 9 mm to 10 mm. In this variation, the cartridge may form a cup-shaped body 112 with one or more guides 114 defined along the outer surface of the body 112. The guides 114 may be shaped to project from the surface and extend longitudinally to function as guide rails as the cartridges are translated through the cleaning device during use. The number of guides 114 may vary from a single guide to two or more guides where the guides are positioned along opposite sides of the body 112. The body 112 may form a receiving cavity 116 within which a cleaning element 118 may be placed. The cleaning element 118 used may be configured in any of the variations described herein although the embodiment shown may also include an instrument receiving cavity 120 which may optionally be configured into a number of different cavity shapes, e.g., star-shaped, for optimally receiving and contacting the instrument for cleaning. An optional removable cap or cover 122 may also be included. Moreover, the cleaning element 118 may remain compressed within the cavity 120, as shown in FIG. 6A, when the cap 122 is in place. With the cap 122 removed, the cleaning element 124 may reconfigure into its expanded shape 124 to extend beyond the cartridge body 112 for cleaning, as shown in FIG. 6B.

FIG. 6C illustrates an exploded assembly view of the cartridge having a floor 132, e.g., microfiber film or base, which may be positioned beneath the cleaning element 118. The cartridge may optionally utilize the cap 122 or a tabbed flexible covering 126 having a tab 128 which may be attached 130 along a side portion of the covering 126.

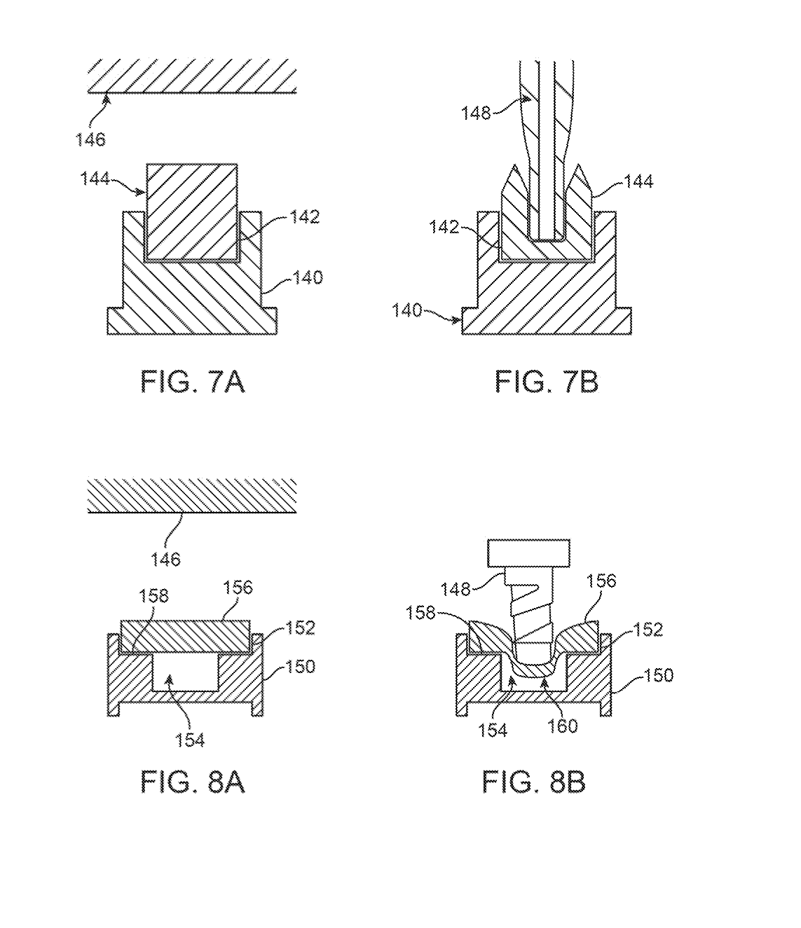

The cleaning element itself may also be configured in various shapes or may be configured to interact with the various cartridge bodies in predetermined ways depending upon the type of surface or instrument to be cleaned. FIGS. 7A and 7B show cross-sectional side views of one variation of the cleaning element 144 which may be configured for attachment along a uniform surface of the cartridge within a receiving cavity 142 and which extends distally from the cartridge body 140. With the cleaning element 144 configured into, e.g., a tubular shape, the cleaning element 144 may be used to contact and clean any flat surface 146 (including a skin surface). The cleaning element 144 may also be used to clean an instrument 148, e.g., IV port, luer fitting, etc., which may be pressed into the cleaning element 144 which may collapse or at least partially envelope the instrument 148 for cleaning not only the distal surfaces of the instrument 148 but also its side surfaces.

In another variation, the cleaning element 156 may be positioned to rest upon an annular shoulder 158 above a depression 154 defined within the receiving cavity 152 of cartridge body 150, as shown in the cross-sectional side view of FIG. 8A. The cleaning element 156 may still be used to clean any flat surface 146 but the addition of the depression 154 may allow for the cleaning element 156 to deform 160 within the depression 154 to collapse or at least partially envelope the instrument 148, as shown in the cross-sectional side view of FIG. 8B.

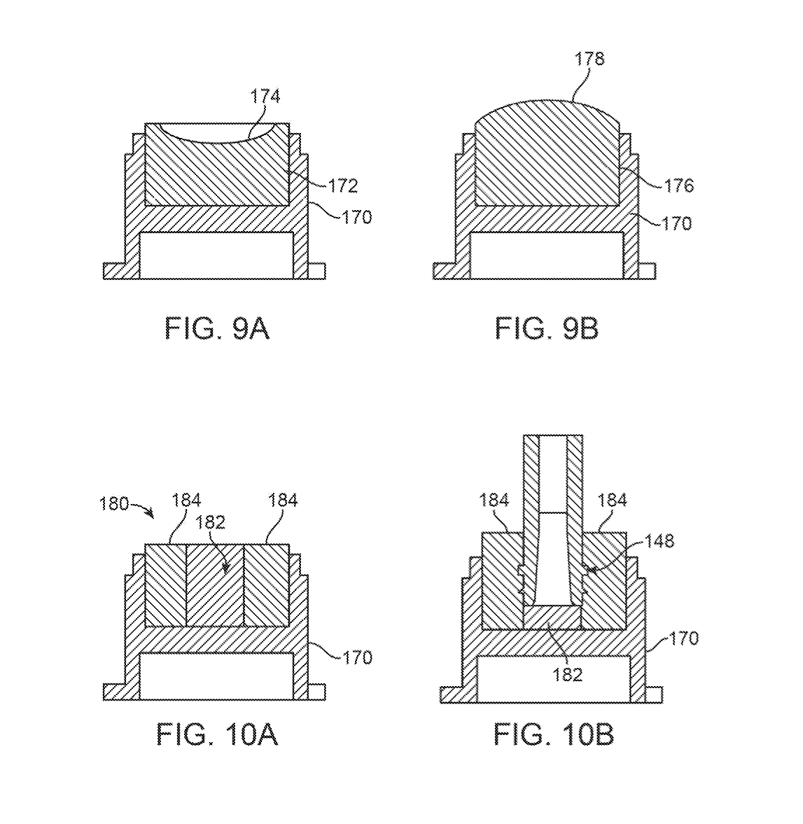

Another variation of the cleaning element is shown in the cross-sectional side views of FIGS. 9A and 9B. The cleaning element 172 may reside within the cartridge body 170 and may also define a concave surface 174 which may extend within the cleaning element 172 to facilitate the cleaning of instruments or surfaces having projections or extending features. FIG. 9B illustrates another variation where the cleaning element 176 may instead define a convex surface 178 which projects arcuately from the cartridge body 170 to facilitate the cleaning of instruments or surfaces having a concave shape or profile.

Yet another variation is illustrated in cross-sectional the side views of FIGS. 10A and 10B which show a cleaning element 180 comprised of a central portion 182 surrounded by an annular portion 184 within the cartridge body 170. This variation may be suited to receive an instrument 148 within the cleaning element 180 by collapsing the central portion 182 and allowing the sides of the instrument 148 to become completely enveloped by the interior surfaces of the annular portion 184. As previously mentioned, the annular portion 184 may define any number of shapes for receiving an instrument 148 and the annular portion 184 may also be configured to be fully circumferential or just partially circumferential with respect to the central portion 182.

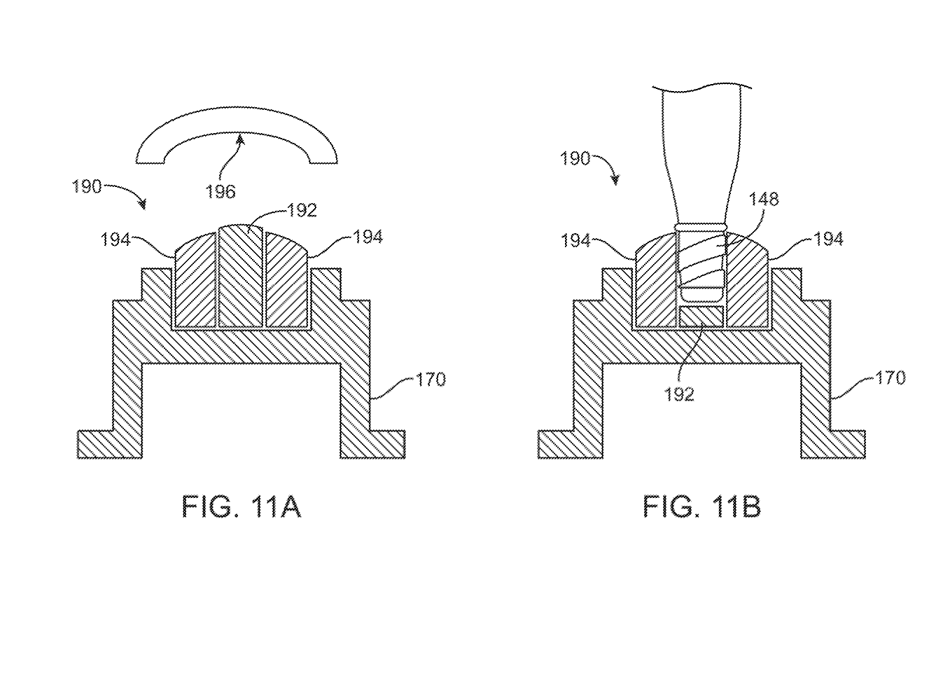

In yet another variation, the cleaning element 190 may be configured to have a central portion 192 having a convex surface as well as an annular portion 194 having a convex surface to collectively form the cleaning element 190, as shown in the cross-sectional side view of FIG. 11A. This configuration may not only allow the cleaning element 190 to clean concave surfaces 196 but may also allow for the cleaning of instruments 148 which may be inserted within the cleaning element 190 such that the annular portion 194 may clean the sides of the instrument 148, as shown in FIG. 11B.

Regardless of the cleaning element configuration, any of the variations described may be used with any of the cartridge body variations also described. Moreover, while each of the cartridges within a cleaning device may be uniform, any of the various configurations of the cartridges as well as cartridges having any of the various configurations of the cleaning elements may be used in any number of combinations or any order of cartridges within a single cleaning device, if so desired.

Cartridge Advancement

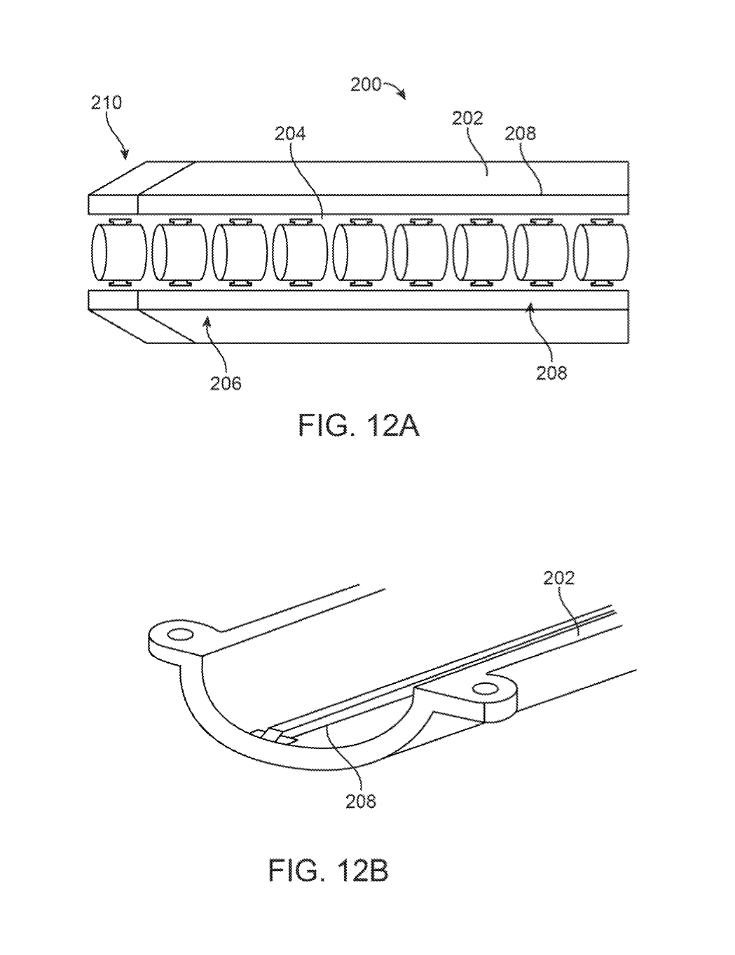

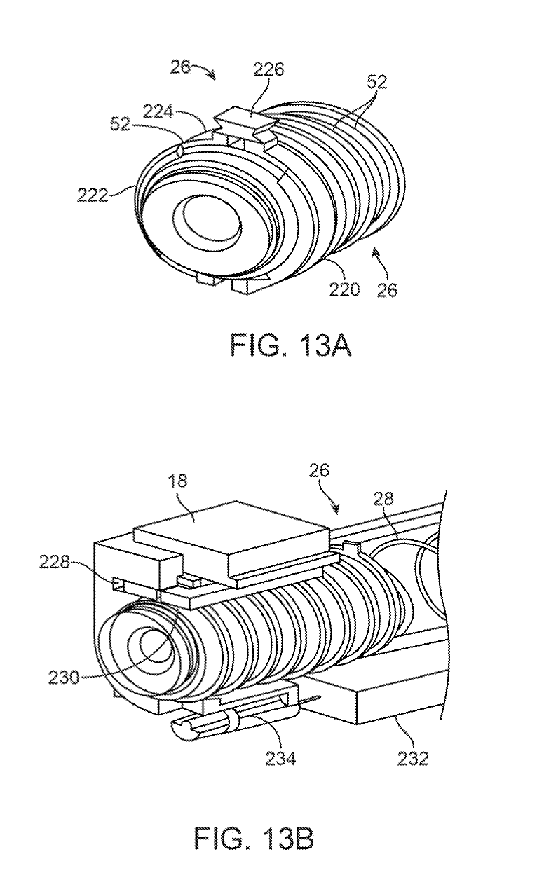

Turning now to the advancement mechanism for securing, ejecting, and advancing the cartridges within the cleaning device, FIG. 12A illustrates generally a partial cross-sectional representation of a cleaning device 200 comprised of a body 202 and a channel 206 defined therethrough for holding the one or more cartridges 204. This example illustrates one or more guide rails 208, which are also shown in the detail perspective view of FIG. 12B, which may be defined along the channel 206 for guiding the cartridges during translation and for maintaining an orientation of the cartridges relative to the device. Generally, the advancement mechanism 210 may be located near or at the distal end of the cleaning device 200 in proximity to the distal opening through which the cartridges are positioned for use and ejected from.

FIG. 13A illustrates a perspective view of one variation of an advancement and retaining mechanism which may be used in the cleaning device. This variation utilizes a circularly shaped ring-like retaining member 220 which has at least one gap or split 224 positioned in proximity to the advancement mechanism 226. An optional gap or split may also be defined on the opposite side of the retaining member 220 as well. When in its secured configuration, the retaining member 220 may be urged or biased to towards one another such that the retaining member 220 may close upon the retaining lip 222 defined circumferentially around the terminally positioned cartridge to allow for the terminal cartridge to be used for cleaning. Pressing upon the advancement mechanism 226 may force the retaining member 220 away from one another allowing for the retaining lip 222 to advance distally past the retaining member 220 such that the terminal cartridge is ejected.

FIG. 13B illustrates a perspective view of another variation of the advancement and retaining mechanism. In this variation, two apposed release arm members 228, 230 may be positioned in proximity to the advancement mechanism 18 such that the arm members 228, 230 extend longitudinally and are biased towards one another. In their securement configuration, the arm members 228, 230 may latch upon or against the lip of the cartridge or a tab projecting from the cartridge body or a cap (as described herein). When secured, the terminal cartridge projecting from the distal opening of the cleaning device may be used for cleaning. When the advancement mechanism 18 is depressed, the arm members 228, 230 may be forced apart from one another to release the cartridge lip or tab and thus allowing for the cartridge to be advanced distally as urged by the cartridge actuation 28. A controller and/or processor 232 may be incorporated into the cleaning device for providing various controls or monitoring features as well as a motor or actuator 234 in proximity to the cartridges (as described in further detail herein).

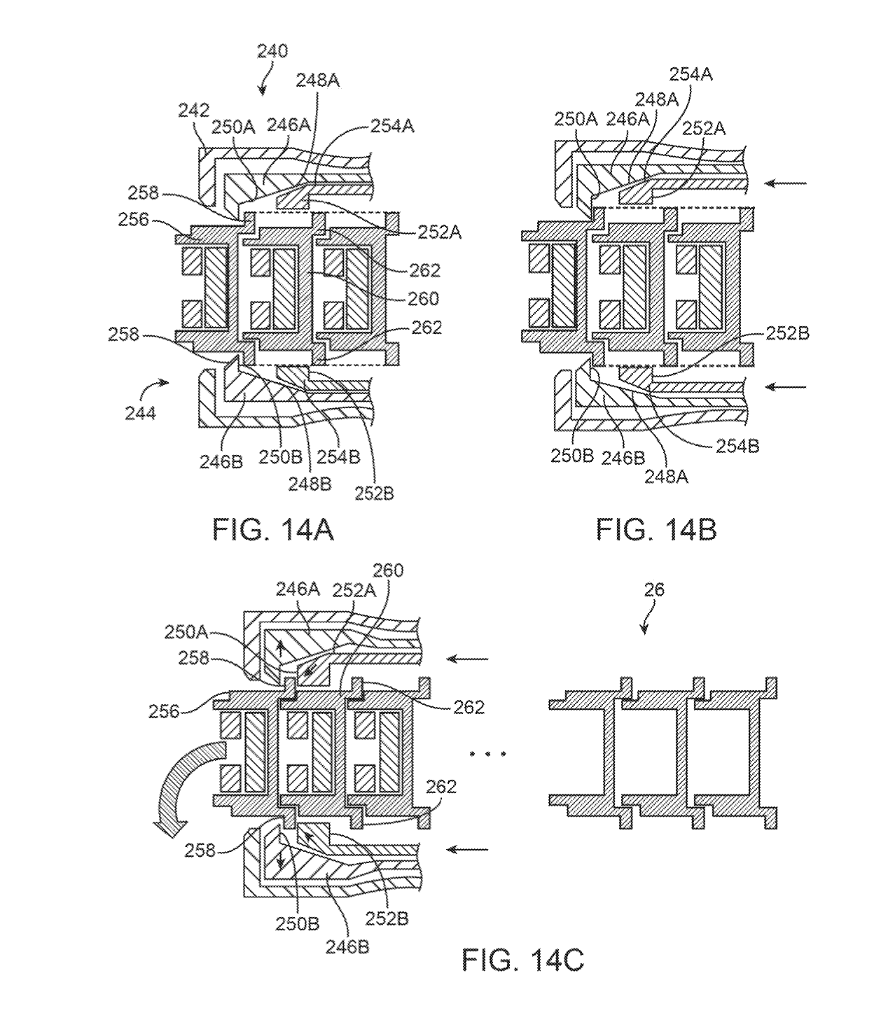

FIGS. 14A to 14C illustrate cross-sectional side views of another variation of an advancement and retaining mechanism 240. The housing 242 of the cleaning device is shown and the distal opening 244 may be seen through which the terminal cartridge is positioned for use. At least two retaining arm members 246A, 246B may be positioned on opposite sides of the terminal cartridge, e.g., first cartridge body 256, such that each arm member defines a retaining shoulders 250A, 250B extending transversely and towards the retaining lip 258 of the first cartridge body 256. Each arm member may also define an angled inner surfaces 248A, 248B which is tapered away from the retaining lip 258. Proximal to the retaining arm members 246A, 246B, corresponding release arm members 252A, 252B may be positioned between the retaining lip 258 of the first cartridge body 256 and the retaining arm members 246A, 246B. Each of the release arm members 252A, 252B may define an angled outer surfaces 254A, 254B.

Once the advancement mechanism 18 is depressed, each of the release arm members 252A, 252B may be urged distally through the cleaning device and towards the retaining arm members 246A, 246B. As the angled outer surfaces 254A, 254B of the release arm members 252A, 252B contact against the angled inner surfaces 248A, 248B of retaining arm members 246A, 246B, as shown in FIG. 14B, the retaining arm members 246A, 246B may be forced radially outward away from the first cartridge 256 such that the retaining lip 258 becomes disengaged from the retaining shoulders 250A, 250B. This disengagement allows for the first cartridge 256 to be ejected through the distal opening and for the second cartridge 260 to be advanced distally into position within the distal opening. As the retaining arm members 246A, 246B are forced radially outward, the release arm members 252A, 252B are simultaneously forced radially inward against the body of the second cartridge 260 and against the second cartridge lip 262 to prevent the second cartridge 260 from also being ejected, as shown in FIG. 14C. Retraction of the release arm members 252A, 252B may accordingly allow for the retaining arm members 246A, 246B to reposition themselves into their initial securement position where the retaining shoulders 250A, 250B may now rest against the second cartridge lip 262 to secure its cleaning element within the distal opening of the cleaning device for use. This process may be repeated as desired or needed until the cartridges are exhausted from the cleaning device.

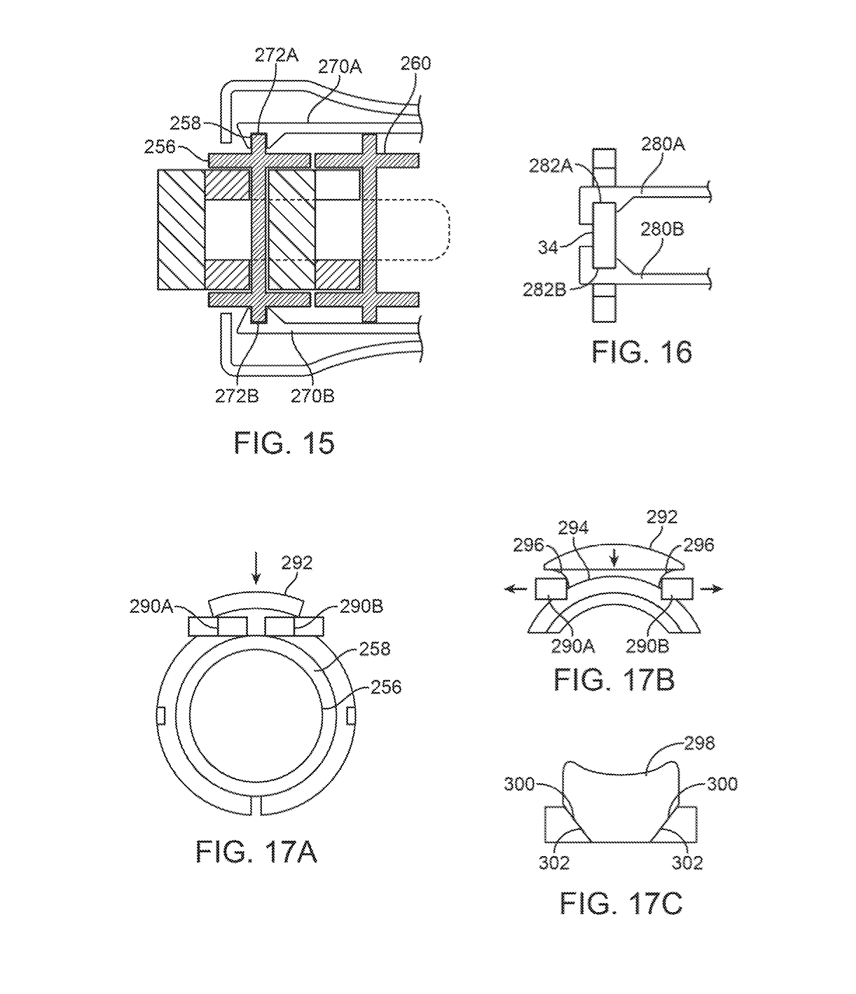

FIG. 15 illustrates a cross-sectional side view of another variation of an advancement and retaining mechanism where release arm members 270A, 270B may be positioned on opposite sides of the cartridges in proximity to the distal opening. The release arm members 270A, 270B may each define cartridge engagement regions 272A, 272B which are shaped to engage the retaining lip 258 of first cartridge 256. When the advancement mechanism 18 is depressed, each of the release arm members 270A, 270B may be forced radially outward away from the retaining lip 258 to disengage the first cartridge 256 which may be ejected. In another variation, rather than having the release arm members positioned on opposite sides of the cartridges, the release arm members 280A, 280B may instead be positioned on a single side of the cartridges such that the cartridge engagement regions 282A, 282B are positioned on opposite sides of a projection 34 extending from the cartridge body, as shown in the side view of FIG. 16.

FIGS. 17A to 17C illustrate end views of additional variations for release arm members and advancement mechanisms. As shown in FIG. 17A, the release arm members 290A, 290B may be positioned along a single side of the cartridge 256 (as described herein) adjacent to the advancement mechanism 292. When the advancement mechanism 292 is depressed, a tapered pushing member 294 having tapered interfaces 296 defined along both sides and extending from the mechanism 292 may force the release arm members 290A, 290B apart from one another, as shown in FIG. 17B. As the arm members 290A, 290B draw apart the cartridge 256 may be released to advance distally for ejection from the cleaning device. An alternative advancement mechanism 298 is shown in the end view of FIG. 17C illustrating a contoured member having tapered interfaces 300 for sliding contact against the corresponding release arm tapered interfaces 302 to urge the arms apart from one another.

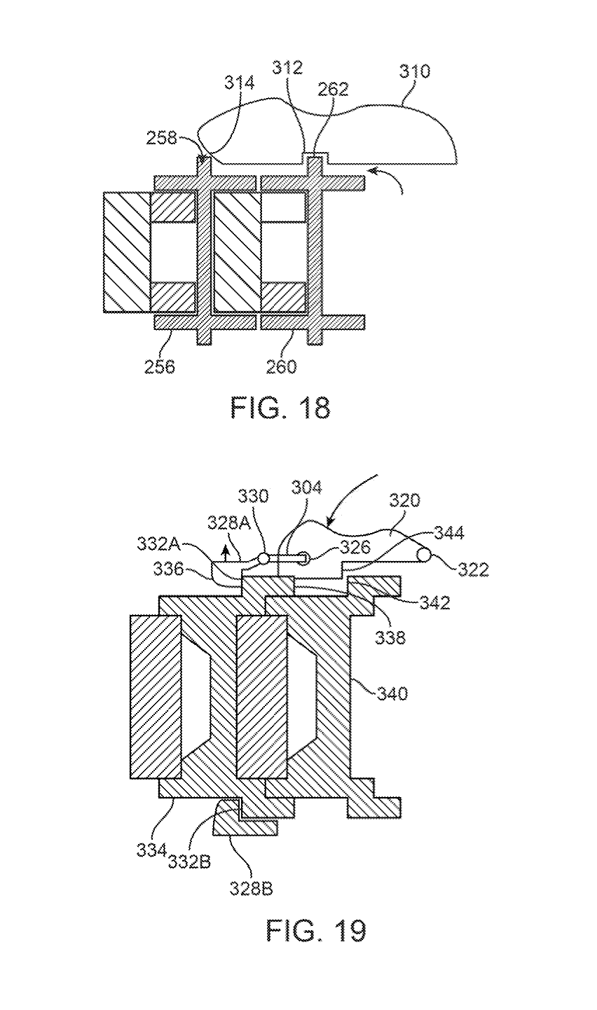

FIG. 18 shows a partial cross-sectional side view of yet another variation of an advancement mechanism 310 which defines a cartridge engagement region 312 sized to engage the retaining lip 262 of the second cartridge 260 while pushing distally against the retaining lip 258 of the first cartridge 256. The mechanism 310 may define an angled surface 314 located along the distal edge of the mechanism 310 to push distally against the retaining lip 258 to eject the first cartridge 256 from the cleaning device. Once the first cartridge 256 has been ejected, the mechanism 310 may be disengaged from the retaining lip 262 allowing for the distal advancement of the second cartridge 260.

FIG. 19 illustrates a partial cross-sectional side view of another variation of an advancement mechanism 320 which may be restrained via a pivot 322 located along a proximal portion of the mechanism 320 to move in an angled pathway. A distal portion of the mechanism 320 may be attached to a coupling member 324 via a pivot 326 and the coupling member 324 may extend to form a release arm member 328A. A corresponding release arm members 328B may be located on an opposite side of the cartridges. The release arm member 328A itself may be restrained via a pivot 330 along its length. As the mechanism 320 is depressed, the coupling member 324 may force the release arm member 328A to move radially away from the cartridges via pivot 330 such that a proximal engagement surface 332A of release arm member 328A and a corresponding proximal engagement surface 332B of release arm member 328B are angled away from the cartridges to release the retaining shoulder 336 of first cartridge 334. Simultaneously, a proximal shoulder 344 defined along the mechanism 320 may abut the shoulder 342 of second cartridge 340 to prevent its distal movement while also pushing against the proximal surface 338 of the first cartridge 334 to urge the first cartridge 334 in a distal direction for ejection from the cleaning device. Once the mechanism 320 is released, the second cartridge 340 may be unconstrained by the proximal shoulder 344 to then advance distally until the shoulder 342 abuts the proximal engagement surfaces 332A, 332B of the release arm members 328A, 328B.

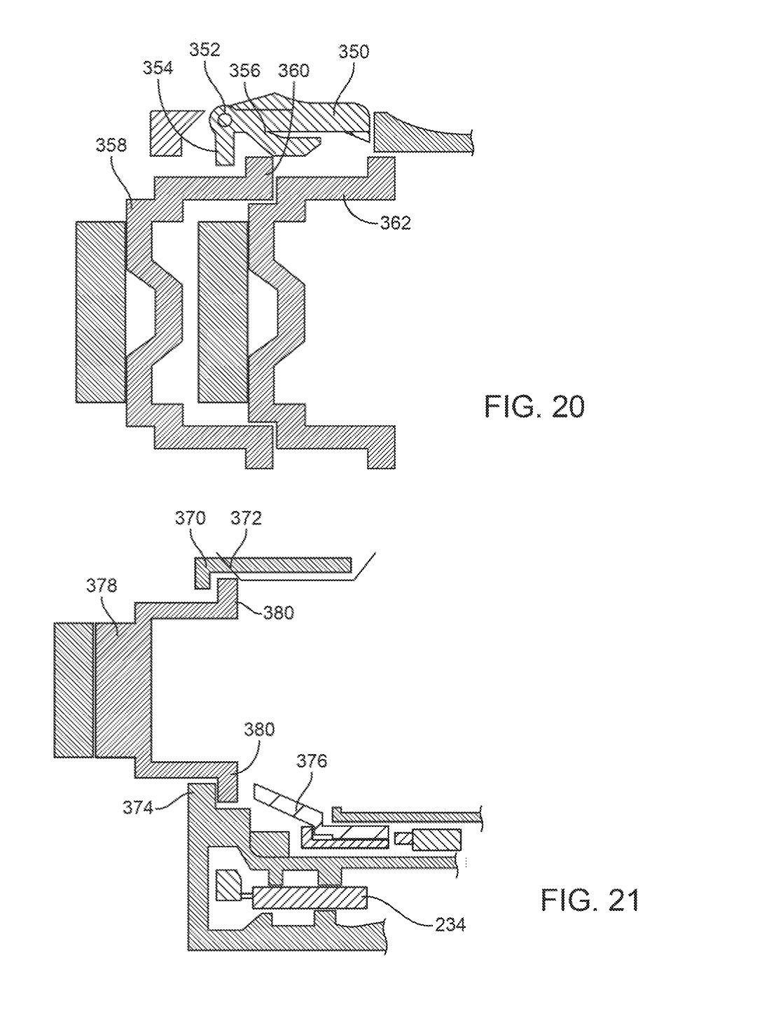

In yet another variation illustrated in the cross-sectional view of FIG. 20, the advancement mechanism 350 may be restrained via a pivot 352 located at its distal end of the mechanism 350. The mechanism 350 may also include a retaining member 354 extending transversely from the pivot 352 and an angled surface 356 defined to extend proximally from the pivot 352. In its engaged state, the retaining lip 360 of the first cartridge 358 may be secured between the retaining member 354 and angled surface 356. However, when the mechanism 350 is actuated, the mechanism 350 may rotate about pivot 352 such that the angled surface 356 may push distally against the retaining lip 360 while simultaneously disengaging the retaining member 354 to allow for the retaining lip 360 to advance distally. Because the angled surface 356 is rotated inwardly against the second cartridge 362, the second cartridge 362 is prevented from advancing further distally until the mechanism 350 is released whereby the second cartridge 362 may then become engaged against the retaining member 354 ready for use.

Another variation is shown in the partial cross-sectional side view of FIG. 21 which illustrates a retaining member 370 adjacent the angled surface 372 of a release mechanism. The retaining lip 380 of cartridge 378 may be restrained by the retaining member 370 and retaining member 374. A pusher mechanism 376 may be urged distally to release the retaining lip 380 from the retaining members 370, 374 when actuated.

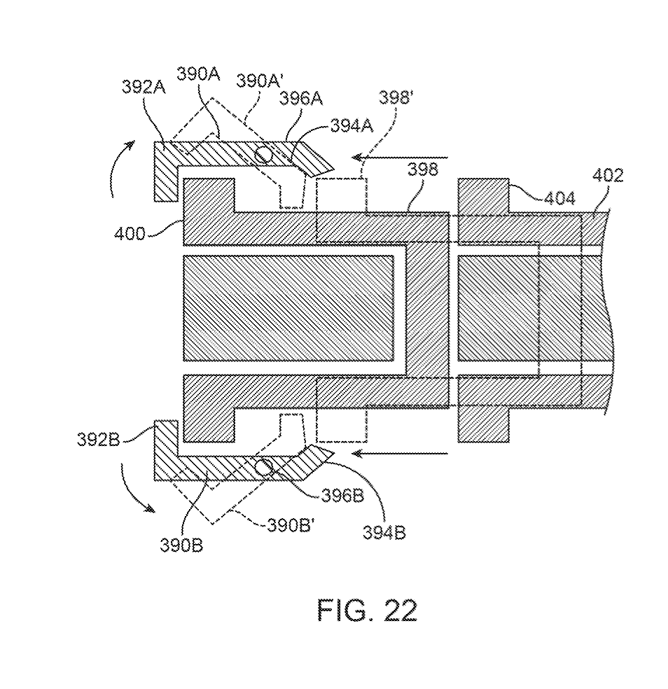

Yet another variation is illustrated in the side view of FIG. 22 where release arm members 390A, 390B may each have retaining members 392A, 392B which extend transversely and radially inward as well as angled members 394A, 394B which also extend radially inward at an angle. The release arm members 390A, 390B may be attached via pivots 396A, 396B such that when the release mechanism is depressed, the release arm members 390A, 390B may pivot about each respective pivot 396A, 396B such that the angled members 394A, 394B angle inwardly against the first cartridge 398 and the retaining members 392A, 392B angle outwardly away from the first cartridge 398.

In this variation, the cartridges may be formed to have a body which retains the cleaning element within the cartridge and the retaining lip 400 is formed annularly around the distal edge of the cartridge, as shown. With this cartridge configuration, once the retaining members 392A, 392B angle outwardly into their pivoted release arms 390A′, 390B′ configuration, the retaining lip 400 may be disengaged to advance distally for ejection. The angled members 394A, 394B may angle inwardly to engage the retaining lip 404 of the second cartridge 402 to prevent further advancement of the second cartridge 402 from the cleaning device.

Regardless of the variation of the release mechanism, any of these variations described is fully and expressly intended to be utilized with any of the cartridge variations described herein and in any number of combinations, as practicable, and furthermore is not limited to any particular combination of features.

Cleaning Enhancement

In any of the variations described, a motor or actuator 234 in communication with at least the terminal cartridge may be optionally incorporated for providing motion to the cleaning element. This motion may enhance the cleaning action of the cartridge and cleaning element when contacted against the surface and/or instrument to be cleaned. A controller and/or processor 232 may also be incorporated into the cleaning device for providing various controls or monitoring various features as well as controlling the motor or actuator 234.

The controller and/or processor 232, for instance, may be programmed to track usage time, location of usage, the power used for the cleaning device as well as other parameters such as the time spent on cleaning particular instruments. The controller and/or processor 232 may also be programmed to track which patients the cleaning device has been assigned to and/or which practitioner has utilized the device as well as parameters such as the physical location of the cleaning device. Additionally, the cleaning device may further include memory capacity as well as wireless transmission and reception capabilities. Hence, the controller and/or processor 232 may be electrically coupled to on-board memory for storing various parameters (e.g., for downloading) or for wirelessly transmitting these parameters to a remote location such as a server through various methods, e.g., Bluetooth®, radio signals, infrared, etc. where the data may be viewed or processed by another party remotely, e.g., through access over an internet based cloud storage system. Additionally, the controller and/or processor 232 may also be programmed to time the cleaning of an instrument. For instance, the controller and/or processor 232 may be programmed to actuate the cleaning element for a minimum period of time, e.g., 1 to 15 seconds or more, once an instrument has started cleaning to provide for sufficient disinfection. The period of time may be preset to any length of time.

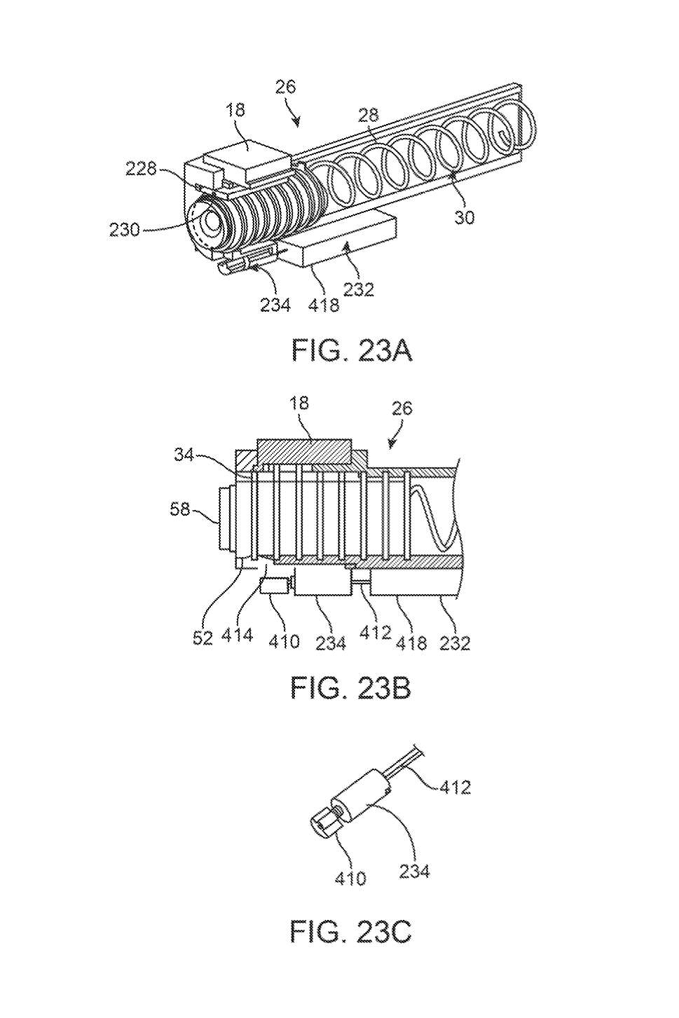

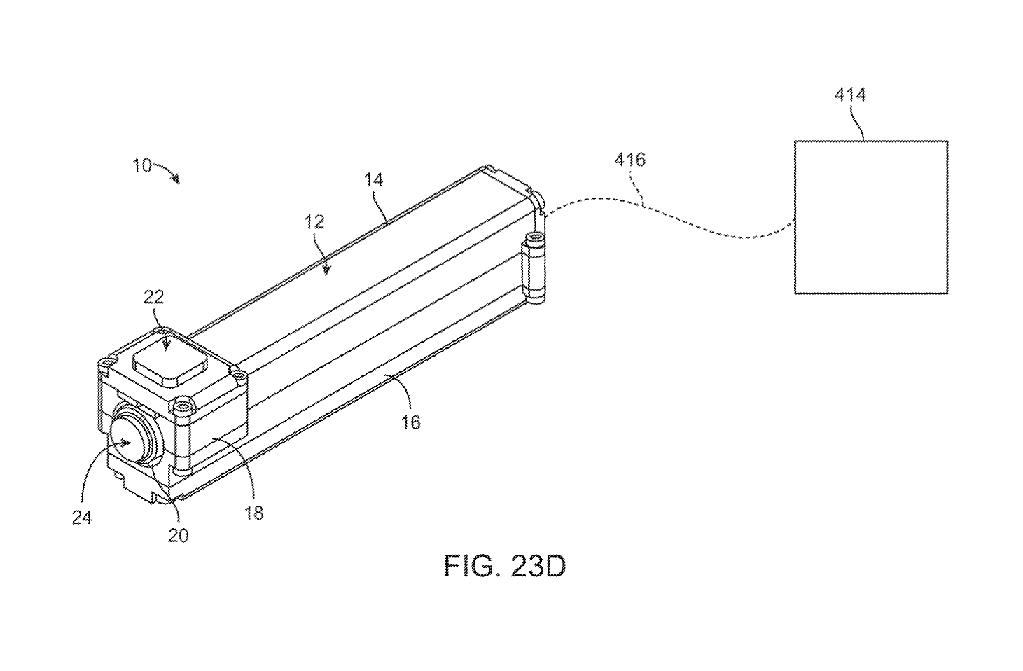

FIGS. 23A and 23B illustrate perspective and side views of one variation of a cleaning device having an actuator 234 integrated into the cleaning device. The application of vigorous friction to the instrument being cleaned may achieve a significant logarithmic reduction of surface bacterial colony loads. The actuator 234 may comprise a motor in electrical communication, e.g., via wire 412, with controller and/or processor 232. In this variation, a mass element 410 may be eccentrically coupled to the actuator 234 such that activation of the actuator 234 may rotate the mass element 410 which imparts a vibrational motion or pulses to the actuator 234 and to a vibrational conductor 414. The vibrational motion may be transmitted via the vibrational conductor 414 which may comprise any vibrationally conductive material such as a metal or plastic to conduct the vibrations into contact against the terminally positioned cartridge positioned at the distal opening, as shown in the side view of FIG. 23B. The vibrational conductor 414 may thereby conduct the vibrations such that the cleaning element 58 positioned at the distal opening may vibrate or move accordingly to enhance the cleaning action. The distal portion of the conductor 414 may be tapered such that once the terminal cartridge has been ejected, the subsequent cartridge may be advanced past the conductor 414 while maintaining contact for vibrational conduction.

A perspective view of an actuator 234 and mass element 410 is illustrated in FIG. 23C. The actuator 234 may be interchangeably removed from the cleaning device and may be configured into various shapes to accommodate incorporation into the cleaning device. Moreover, the controller and/or processor 232 may be programmed or configured to control parameters such as timing, vibrational frequency, etc. of the actuator 234 as well as receive feedback from the actuator 234. The actuator 234 may be activated via the controller and/or processor 232 when the user manually operates a switch or the actuator 234 may be activated automatically via the controller and/or processor 232 after a predetermined period of time.

In one variation, the controller and/or processor 232 may be programmed to vary the time that the actuator 234 is activated to actuate the cleaning element based on the type of surface or instrument to be cleaned. For instance, if the surface to be cleaned is particularly rough or contoured, the controller and/or processor 232 may be programmed to automatically lengthen the cleaning time for the actuator 234 (e.g., by sensing a relatively large pressure upon the cleaning element as described further below) compared to a cleaning time for a relatively smooth surface to be cleaned.

In order to input various parameters into the cleaning device, variations of the cleaning device may optionally communication through various methods such as RFID, QR codes, two-dimensional bar codes, near field communication, infrared, etc. The cleaning devices may also be optionally stored in a charging or docking station 414 which may provide for charging of power. The station 414 may also include an electronics unit which may be in communication 416, either wireless or wired, with the cleaning device as illustrated in FIG. 23D. Various information or parameters stored by the cleaning device may be downloaded to the docking station 414 which may also be connected via a wireless or wired connection to a remote serve such as a central electronic server system used in a hospital setting.

In yet additional variations, the motor or actuator 234 may be activated manually or it may be activated automatically when the cleaning element has a predetermined pressure applied to it so that the amount of force and friction remains consistent in cleaning the surface or instrument. The controller and/or processor 232 may be programmed to activate the actuator 234 when a predetermined amount of force is sensed by the terminally positioned cartridge pressing against the release arms or against a pressure sensor 418 in proximity to the cartridges, i.e., when the detected pressure exceeds a predetermined threshold level. After the cleaning element has been actuated for a predetermined period of time, which may be controlled by the controller and/or processor 232, once the pressure is released (e.g., when the device is removed from the surface or instrument being cleaned) and the sensed pressure falls below the predetermined threshold level, the pressure sensor 418 may send a signal to the controller and/or processor 232 which may then activate the release mechanism to automatically eject the used cartridge so that the next cartridge may be advanced ready for use or ready to be unsealed.

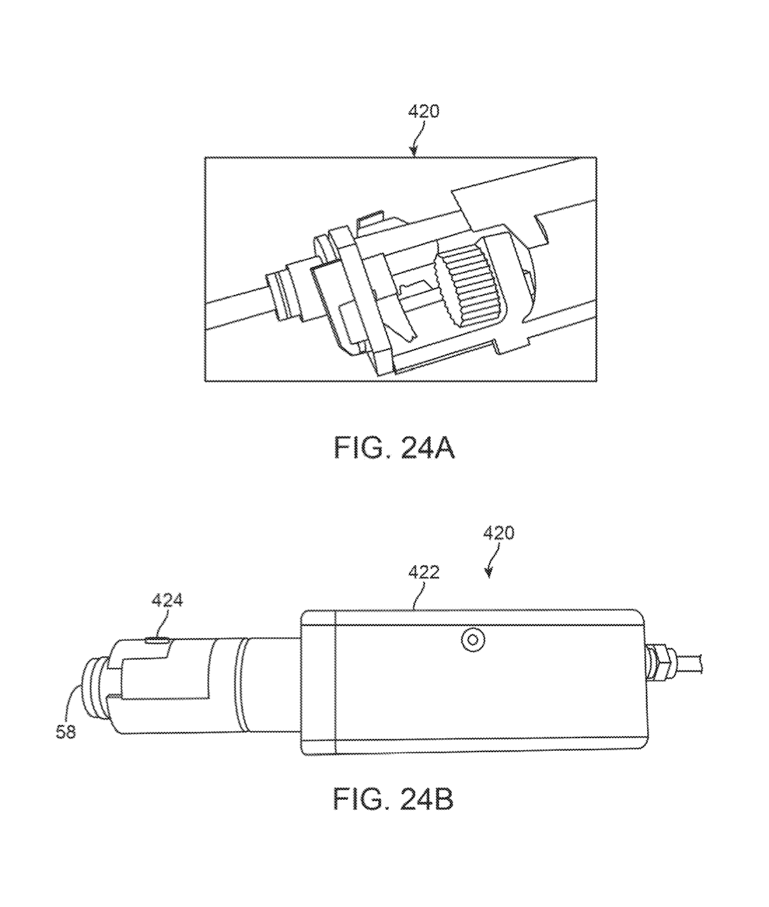

FIG. 24A and 24B illustrate perspective and side views of another variation for a rotational oscillation motor 420 which may function as the actuator. This motor 420 may be positioned within a housing 422 while maintaining vibrational conductive contact with the terminally positioned cartridge, as shown. The advancement mechanism 424 may also be seen.

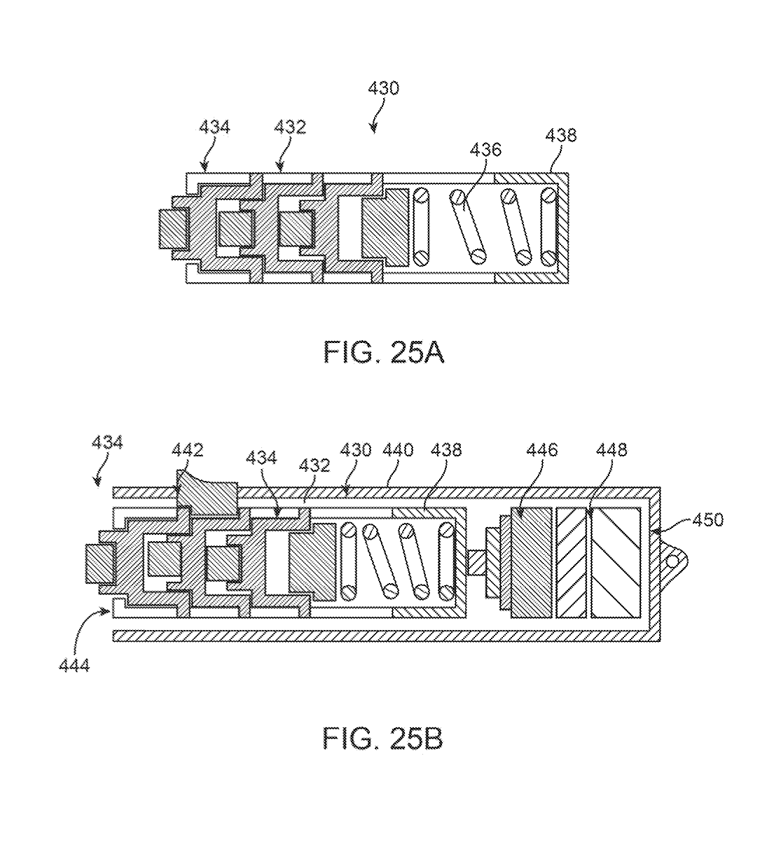

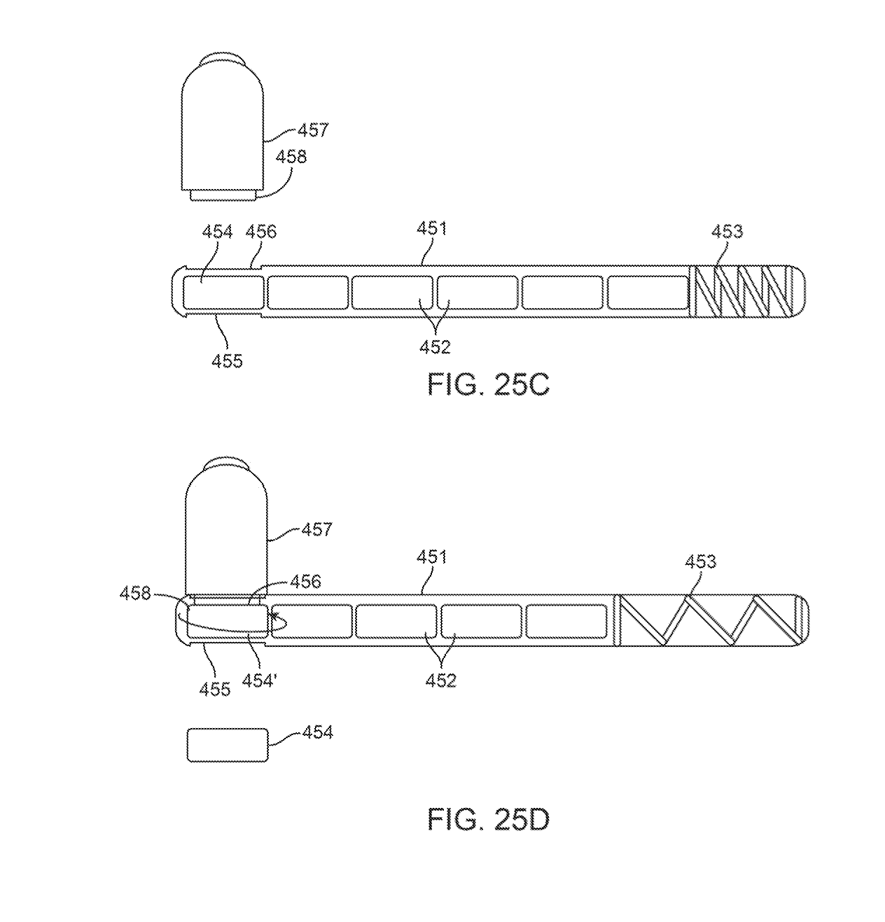

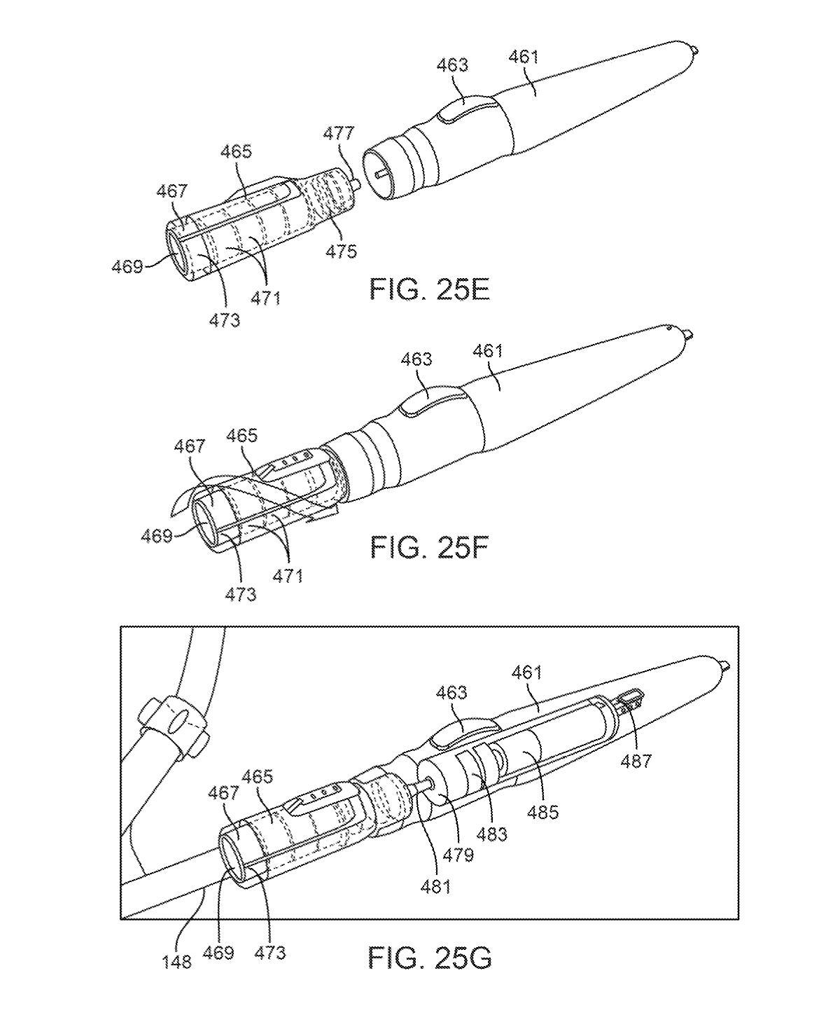

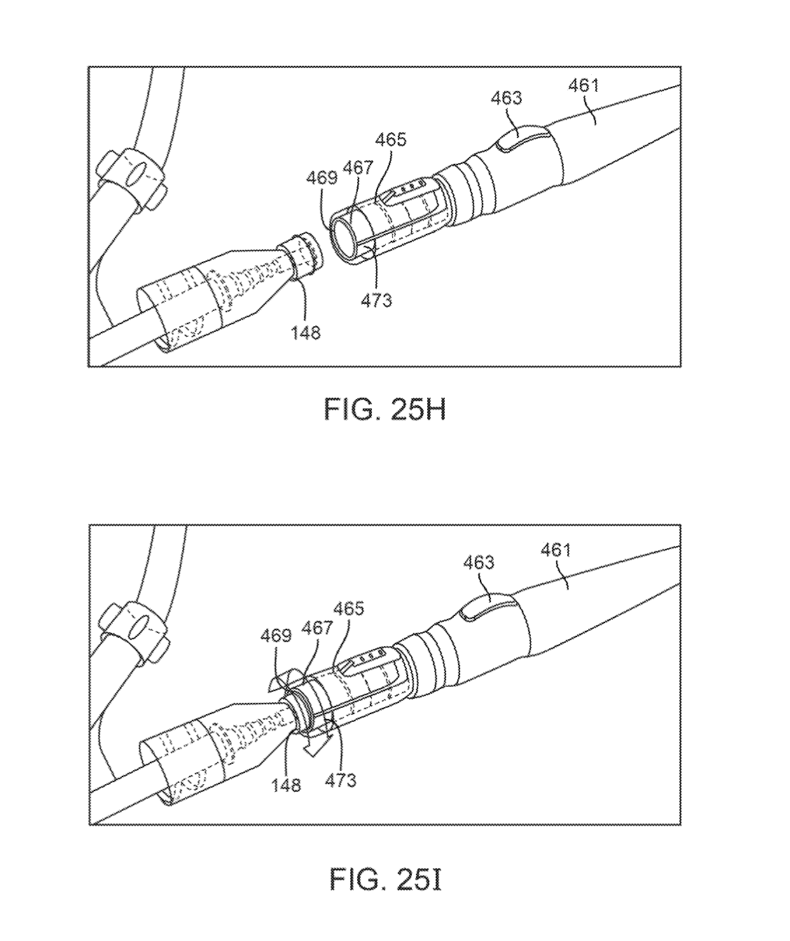

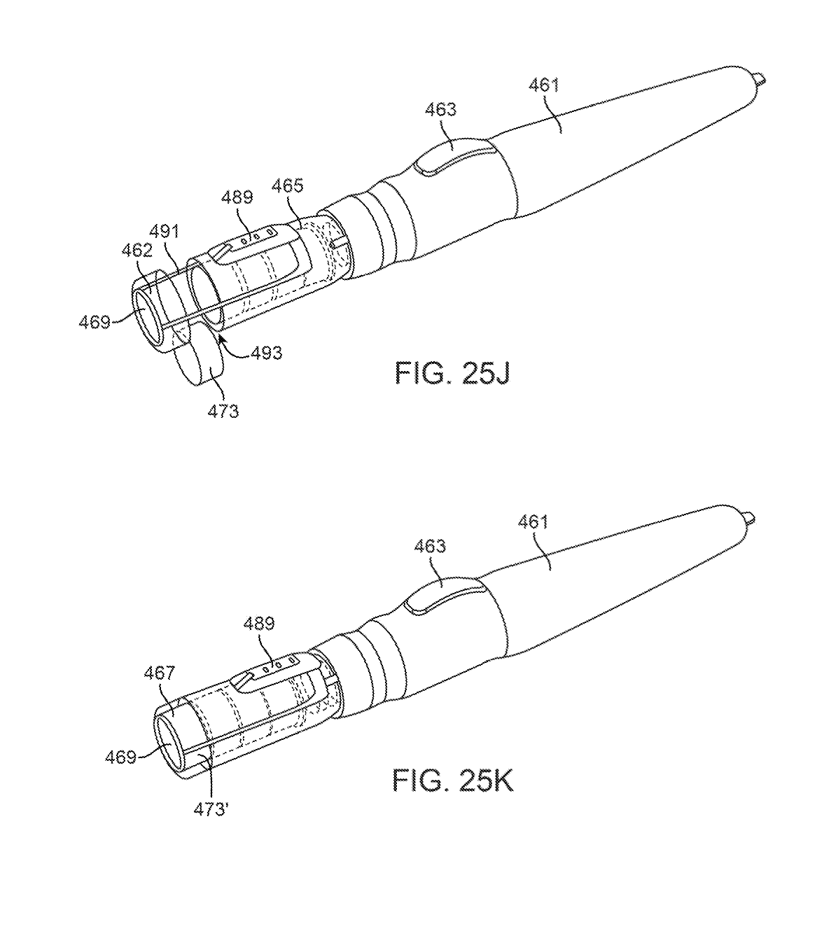

FIGS. 25A and 25B illustrate cross-sectional side views of another variation of a cleaning device which may incorporate a magazine 430. The magazine 430 may comprise an elongate magazine housing 432 which holds one or more cartridges 434 aligned within. The cartridges 434 may have a cartridge actuation mechanism 436, e.g., spring or other biasing member, which may push the cartridges 434 distally through the magazine housing 432. A proximal portion 438 of the magazine housing 432 may also be comprised of a vibrationally conductive material. The entire magazine 430 may be removably inserted within the body of the cleaning device 440 as an assembly such that the distal opening of the magazine 432 may align with the distal opening 444 of the cleaning device 440 such that the terminal cartridge is positioned within the distal opening 444 for use. The cleaning device 440 may have an advancement mechanism 442 which may also align and couple with the cartridges 434 held within the magazine 430. The cleaning device 440 may also have an actuation assembly 446, such as a motor, controller and electronics housing 448, and power supply 450 within the cleaning device 440 such that the actuation assembly 446 contacts the proximal portion 438 of the magazine 430 for transmitting vibrations or motion to the terminally positioned cartridge.

FIGS. 25C and 25D illustrate cross-sectional side views of yet another variation of a cleaning device which may align the cartridges side-to-side so that the distal opening for cleaning is located along a side surface of the device. As illustrated in FIG. 25C, the body 451 may similarly have one or more cartridges 452 within with a cartridge actuation 453 (as described herein) located proximally to push each of the cartridges 452 distally within the body 451. In this variation, each of the cartridges 452 are arranged side-to-side so that their cleaning surfaces are facing transversely relative to the longitudinal axis of the device rather than being aligned collinearly. The distal end of the body 451 may have its opening 455 defined along a first side the side of the body 451 while a second opposite side of the body 451 may define an interface 456 through which an actuator and/or controller 457 may be attached for actuating the terminal cartridge 454 positioned adjacent to the opening 455, as shown in FIG. 25C.

Once the surface or instrument to be cleaned is in contact against the cleaning element of the terminal cartridge 454, the actuator and/or controller 457 may be coupled to the terminal cartridge 454 via a cartridge engagement surface 458 to oscillate the cleaning element. After the cleaning has been completed, the terminal cartridge 454 may be ejected through the opening 455 and a second terminal cartridge 454′ may be advanced distally into position adjacent to the opening 455, as shown in FIG. 25D.

In yet another variation, FIGS. 25E to 25K illustrate another variation which may also utilize a magazine which may contain one or more of the cartridges. FIG. 25E illustrates a perspective view of a body 461 of the cleaning assembly having an actuation mechanism 463. The magazine housing 465 may be separate from the body 461 and have one or more cartridges 471 aligned within for use. The magazine housing 465 itself may be comprised of a body having a magazine distal cover 467 slidingly attached. The distal cover 467 may define a distal opening 469 through which the cleaning element of a terminally positioned cartridge 473 may be accessible. The magazine housing 465 may further include the cartridge actuation 475 within as well as a drive shaft interface 477 for providing actuation of at least the terminal cartridge 473.

The magazine housing 465 may be coupled to the body 461, e.g., by a threaded interface, as shown in FIG. 25F, prior to use. FIG. 25G illustrates an assembly view of the internal components of the cleaning device showing how the drive shaft interface 477 may be coupled to a drive shaft 481 contained within the body 461. The drive shaft 481 may be rotating attached to an actuator 479, e.g., a motor, which may in turn be electrically connected to a controller and/or processor 483. A power supply 485 may also be incorporated within the body 461 and a transmitter and/or receiver 487 in electrical communication with the controller and/or processor 483 may also be integrated within the body 461.

With the magazine housing 465 connected, an instrument 148 to be cleaned may be placed within the distal opening 469 into contact with the cleaning element of the terminally located cartridge 473, as illustrated in FIG. 25H. The actuation mechanism 463 may be activated to start the actuator 479 which may turn the drive shaft 481 to provide oscillation or agitation to at least the terminally located cartridge 473 within the magazine housing 465 to enhance cleaning of the of the instrument 148, as illustrated in FIG. 25I.

Once the cleaning of the instrument 148 has been completed, the actuator 479 may be stopped and the distal cover actuator 489 may be activated to urge the magazine distal cover 467 to advance while attached via a connecting member 491. The advancement of the distal cover 467 may create an opening 493 through which the used terminal cartridge 473 may be ejected, as illustrated in FIG. 25J. After the cartridge 473 has been ejected, the distal cover 467 may be slid proximally back in place to close the opening 493. Because the remaining cartridges 471 within the magazine housing 465 are urged distally, each of the remaining cartridges 471 may be translated proximally so that a second terminal cartridge 473′ may be positioned adjacent to the distal opening 469 for use, as illustrated in FIG. 25K.

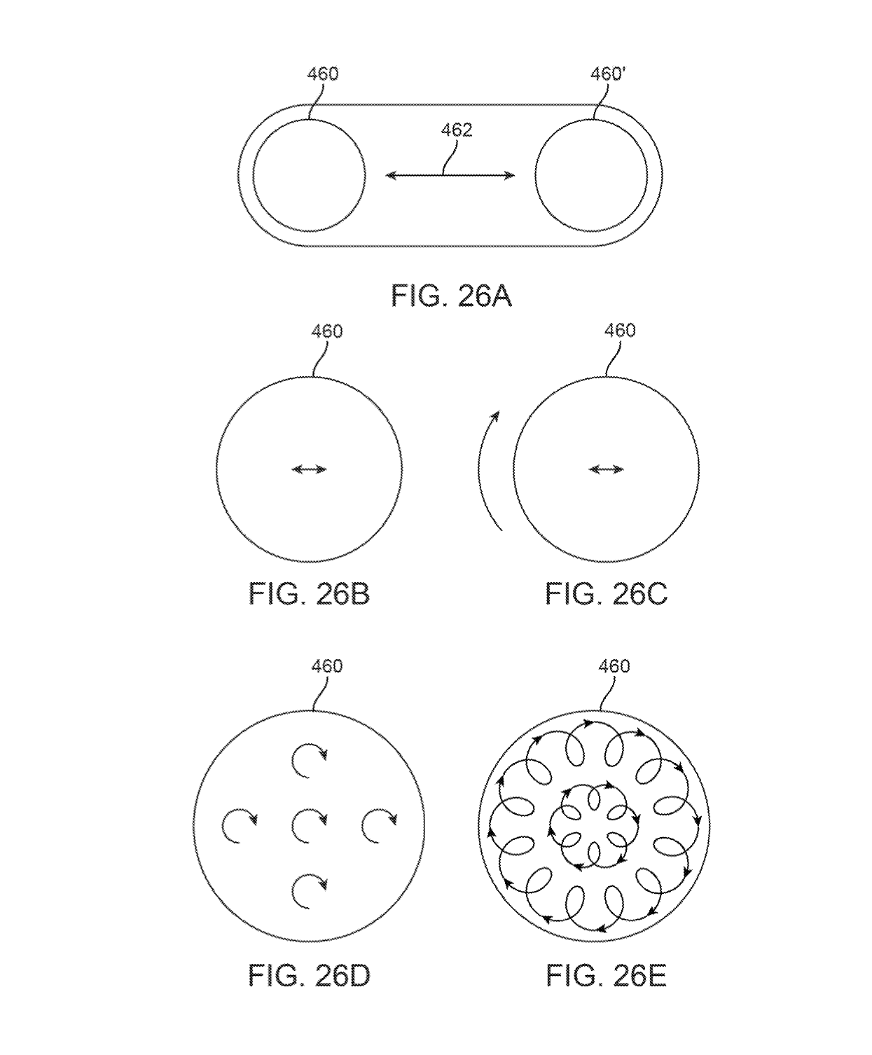

Because the actuator may be configured to impart simple vibrations or any number of motions to at least the terminally positioned cartridge, some of the variations on the motion may be seen in the schematic illustrations of FIGS. 26A to 26E which show an example of a cleaning element 460. The cleaning element 460 may be simply rotated about its longitudinal axis, however, the center of the cleaning element 460 may not present a displacement which provides sufficient cleaning motion. Ideally, the entire cleaning element 460 is displaced from its initial position to ensure sufficient motion to clean any film or bio-agent. The displaced cleaning element 460′ is illustrated as displaced in an exaggerated linear motion 462 shown in FIG. 26A although the cleaning element 460 need not be displaced to such a degree.

FIG. 26B illustrates a simple linear motion of cleaning element 460 while FIG. 26C illustrates both the linear motion in combination with a rotational motion. FIG. 26D illustrates an example of an orbital motion for cleaning element 460 to combine the benefits of the rotational and linear oscillation. FIG. 26E illustrates a random orbital motion for cleaning element 460. Any of these types of motion may be imparted to any of the cleaning elements described herein. Additionally, the processor may also be configured to provide one or more of these types of motions to the cleaning element in a single cleaning device.

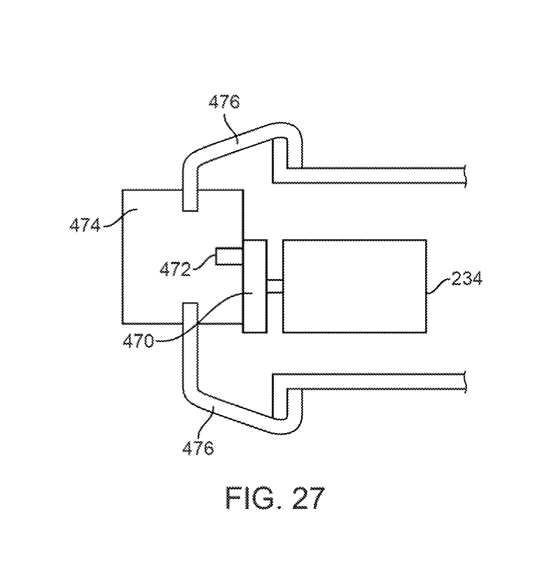

FIG. 27 illustrates a cross-sectional side view of another variation of a cleaning device having a mount 470 for attachment 472 to a coupling head 474 which may be used to hold one or more cartridges. The attachment 472 may be eccentrically coupled to the mount 470 such that rotation of the mount 470 may rotate the coupling head 474 in an orbital or rotational-type motion while a flexible covering 476 may be attached to both the coupling head 474 and to the body of the cleaning device such that coupling head 474 rotates eccentrically relative to the cleaning device while maintaining its orientation.

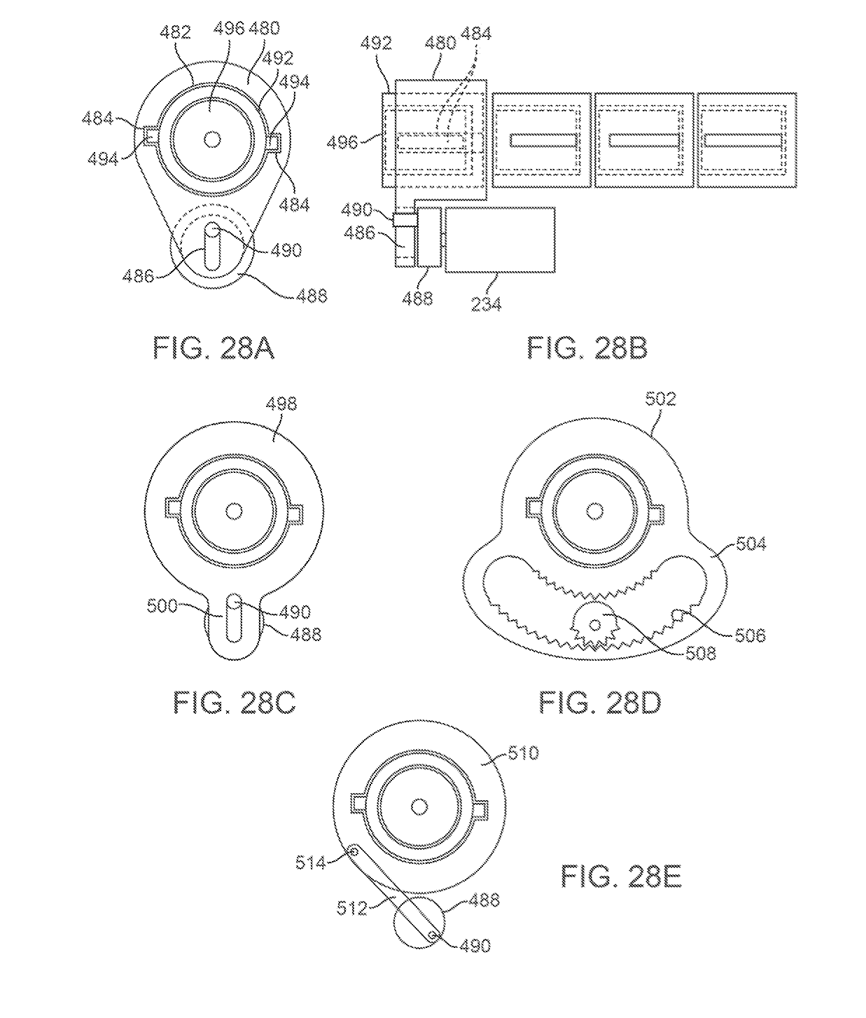

FIGS. 28A and 28B illustrate respective end and side views of another variation of an actuator coupled to the terminally positioned cartridge. A collar 480 may define an opening 482 which is sized to have one or more channels 484 for receiving one or more guides 494 which may be positioned along the cartridges (as described herein). A portion of the collar 480 may project laterally and define a slot 486 extending radially towards the longitudinal axis of the cartridge 492. The slot 486 may be suitably sized for a projection 490 extending from a rotating member 488 rotationally coupled to actuator 234. When the actuator 234 is activated to rotate member 488, the eccentrically positioned projection 490 may rotate about the longitudinal axis of rotating member 488. This rotational movement may force projection 490 to slide within slot 486 which may in turn force collar 480 to oscillate about the longitudinal axis of the cartridge 492 in an alternating rotational manner such that the cleaning element 496 is rotated accordingly. While the collar 480 is shown contacting the terminally positioned cartridge 492, additional cartridges may also be oscillated as well.

FIG. 28C illustrates an end view of another variation of a collar 498 having an extension 500 which defines the slot for coupling with projection 490. FIG. 28D illustrates another variation of a collar 502 which defines a curved extension 504. A curved or arcuate rack gear 506 may be defined around the curved extension 504 for corresponding engagement with a pinion gear 508 coupled to the actuator. As the pinion gear 508 rotates and engages the rack gear 506, the collar 502 may oscillate about the longitudinal axis of the cartridge. FIG. 28E illustrates yet another variation of a collar 510 which is coupled to the rotating member 488 and projection 490 via a connecting member 512. The connecting member 512 may be pivotably coupled to the projection 490 and also to the collar 510 via a pivot 514 such that rotation of the rotating member 488 may force the collar 510 to oscillate rotationally as the connecting member 512 is moved.

Regardless of the actuation configuration, any of the variations described may be used with any of the cartridge body variations and advancement mechanisms described, as practicable. Hence, the variations shown are not intended to be limiting but may encompass any combination of features between the various mechanisms and features.

Covering or Cap

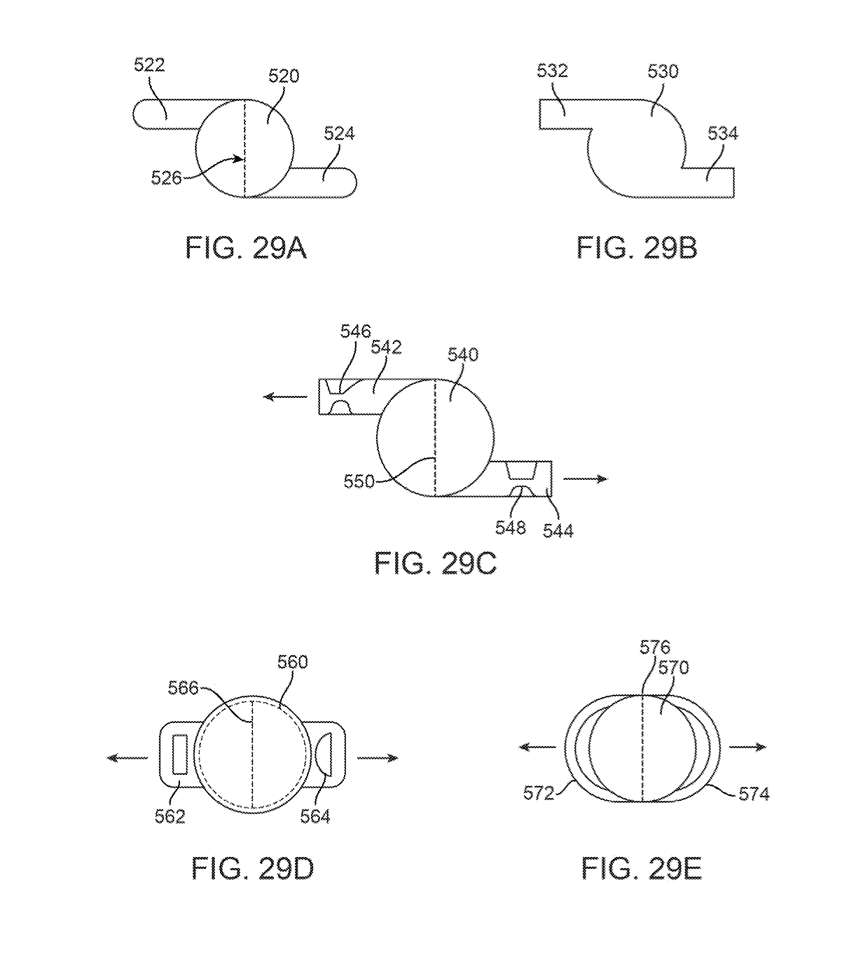

Any of the cartridge variations described may be sealed via an adjacent cartridge. However, any of the cartridge variations may also optionally incorporate an individual covering or seal to maintain sterilization of the cleaning element prior to its use. FIG. 29A illustrates a top view of one variation of a flexible covering 520 which may have a first tab 522 and a second tab 524 which are each positioned to extend tangentially in opposite directions. The flexible covering 520 may be fabricated from various materials, e.g., peelable metal foils, plastic films, wax paper, etc. A tear point 526 (e.g., a weakened portion of the tab 520) may be defined to extend diametrically across the covering 520 and perpendicularly relative to the tabs 522, 524 so that when the tabs are engaged and pulled (e.g., manually or otherwise) in opposing directions, the covering 520 may split along the tear point 526 to expose the cleaning element contained within the cartridge for use. FIG. 29B illustrates another variation of a covering 530 similarly having a first tab 532 and second tab 534 but without the tear point 526 while FIG. 29C illustrates yet another variation of a covering 540 having a first tab 542 and a second tab 544 as well as the tear point 550 but also having one or more engagement features 546, 548 along one or both of the tabs 542, 544 to facilitate engagement of the tabs for pulling.

FIG. 29D illustrates a top view of another variation of a covering 560 having a first tab 562 and a second tab 564 and a diametric tear point 566 although in this variation, the tabs 562, 564 having openings defined therethrough to facilitate their engagement. Similarly, FIG. 29E shows another variation of a covering 570 having a first tab 572 and a second tab 574 as well as a tear point 576 but in this variation, the tabs 572, 574 may define looped structures attached at either end to the covering 570 in proximity to the tear point 576.

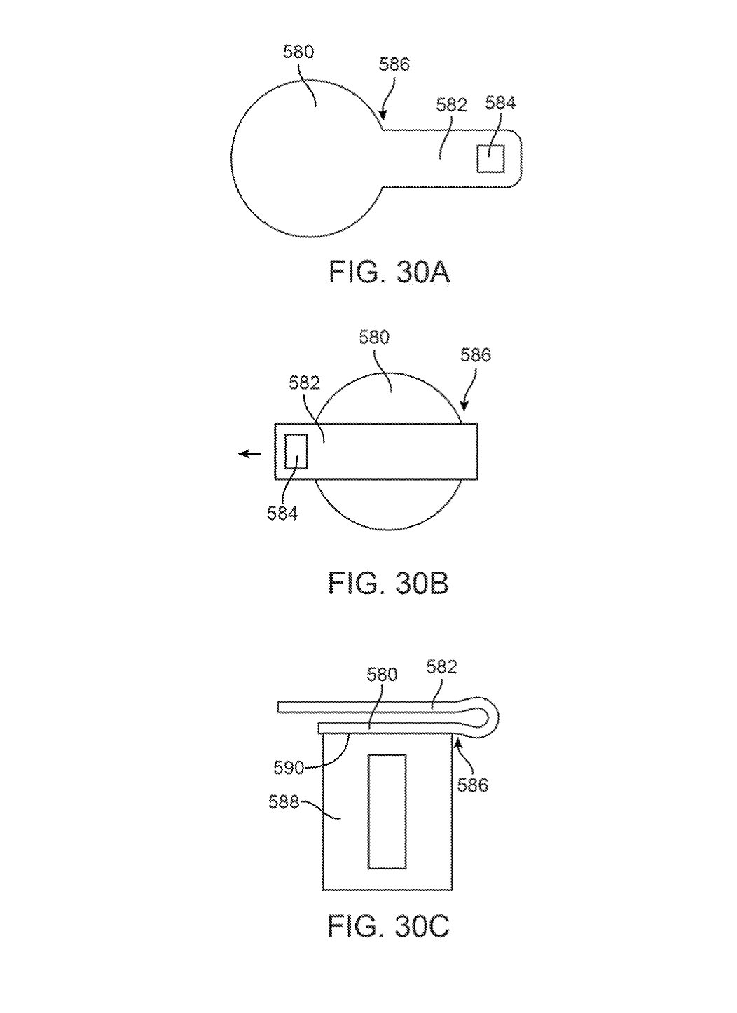

FIGS. 30A to 30C illustrate top and side views of yet another variation of a covering 580 having a single tab 582 which defines an opening 584 at its distal end and has an attachment 586 to the covering 580 at a proximal end of the tab 582. Prior to use, the tab 582 may be folded over upon itself such that it extends back over the covering 580 such that the opening 584 of the tab 582 extends over the cartridge 588 while the covering 584 remains sealed to the cartridge 588.

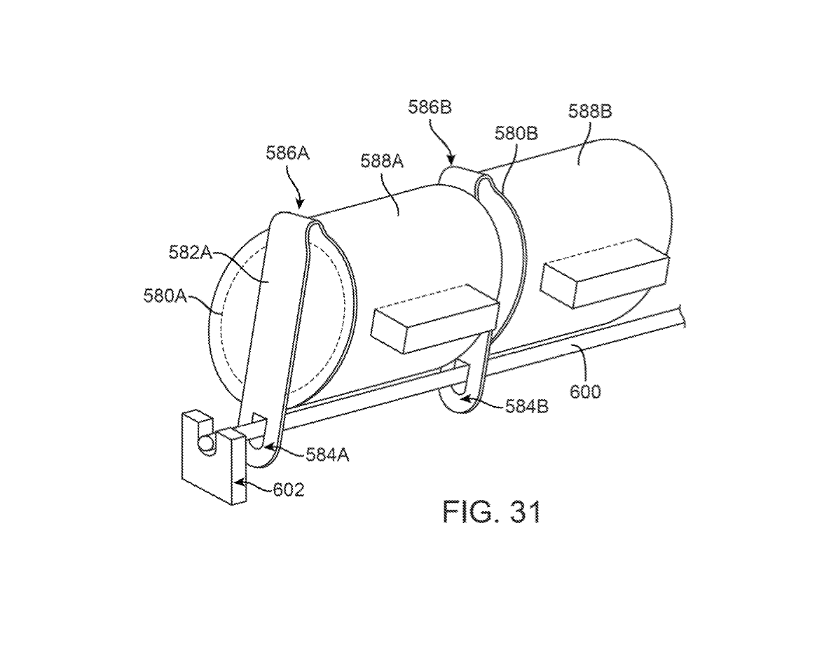

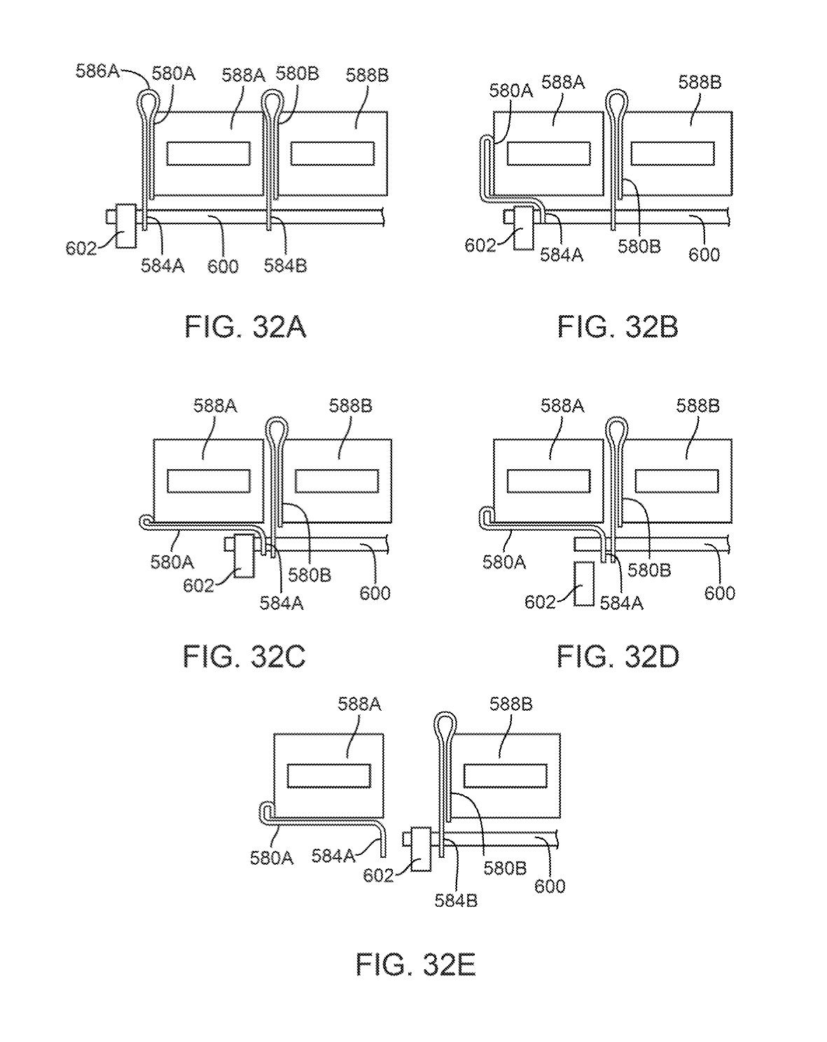

In use, one or more cartridges 588A, 588B having a respective covering 580A, 580B may each be folded over to extend from their attachment points 586A, 586B and over the coverings 580A, 580B such that the openings 584A, 584B of each tab are aligned. A tab rail 600 may be positioned to extend longitudinally through the cleaning device and adjacent to the cartridges such that the tab rail 600 passes through openings 584A, 584B of each tab, as illustrated in the perspective view of FIG. 31. A movable tab stop 602 may be positioned near or at a distal end of the guide rail 600. A side view of the one or more cartridges 588A, 588B with respective coverings 580A, 580B and guide rail 600 passing through each of the openings 584A, 584B may be illustrated in FIG. 32A.

As the cartridges are advanced distally through the cleaning device, each of the cartridges and their respective coverings may advance along the guide rail 600. As the first cartridge 588A is further advanced, the first tab opening 584A may abut against the tab stop 602 but because of the attachment of the tab to the covering 580A, the covering 580A may be forced open as the tab is tensioned by the distal movement of the first cartridge 588A, as illustrated in FIG. 32B. Further distal advancement of the cartridges may further peel the covering 580A from the first cartridge 588A to fully expose the cleaning element within, as illustrated in FIG. 32C. Once the cleaning has been completed with the first cartridge 588A, the tab stop 602 may be retracted from the guide rail 600, as illustrated in FIG. 32D, such that further distal translation of the cartridges may allow for the first cartridge 588A and its covering 580A to be ejected from the cleaning device. The tab stop 602 may be repositioned against guide rail 600 to prevent further advancement of the next tab opening 584B as the second cartridge 588B is advanced for use, as illustrated in FIG. 32E. This process may be repeated for each cartridge having a self-peeling covering.

Any of the covering variations may also be utilized in combination with any of the cartridge variations described herein as well as any of the advancement variations and cleaning enhancement variations as well in any variety of combinations, as practicable. For instance, the covering removal variation shown in FIGS. 32A to 32E may be combined with the actuator variation shown in FIG. 23B and cartridge variation shown in FIGS. 6A and 6B. This is intended to be illustrative of the various combinations possible without being limiting.

Attachments

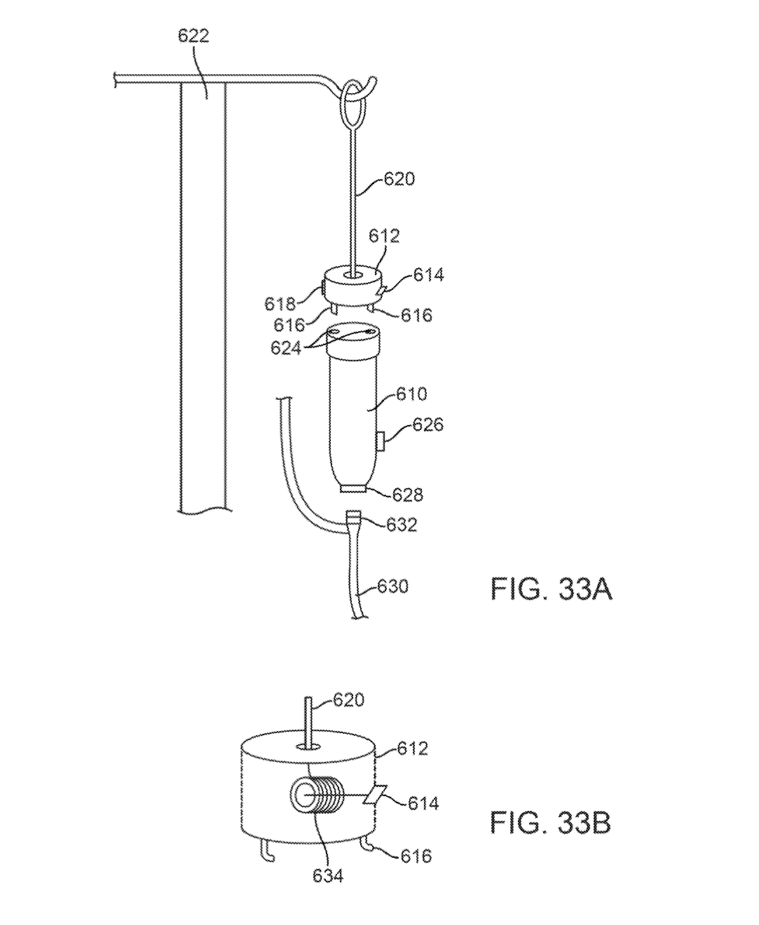

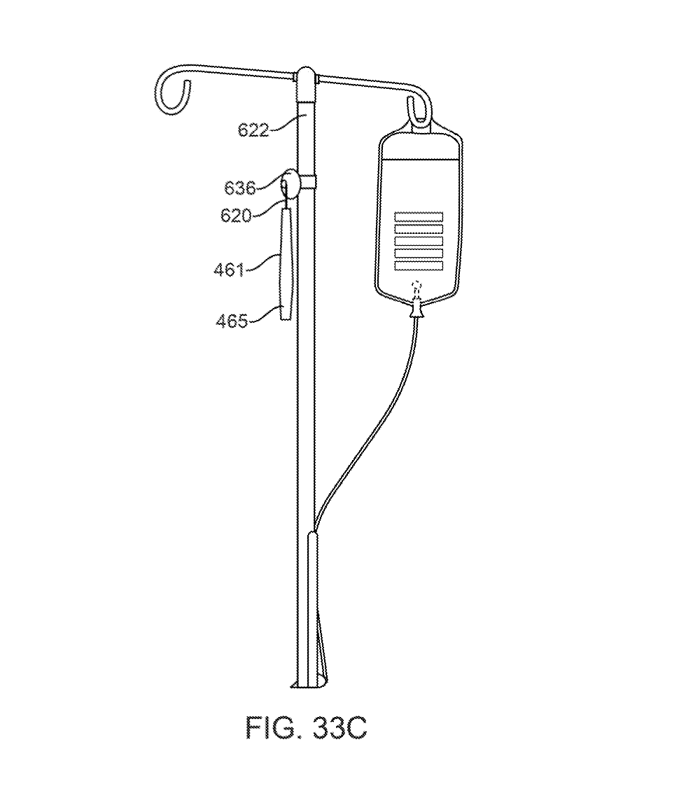

In addition to the various combinations of features, the cleaning device itself may be optionally configured with one or more attachments to facilitate its use. FIG. 33A illustrates a perspective view of one example of a cleaning device 610 which may be optionally attachable to a retractable cable housing 612. The proximal end of the housing of the cleaning device 610 may define one or more attachment points 624 for removable coupling to one or more corresponding connectors 616 extending from the cable housing 612. The cable housing 612 may have a cable 620 retractably housed within upon a cable spool 634, as shown in the perspective view of FIG. 33B, such that the cable 620 may extend, e.g., up to three feet or more, from the housing 612 for coupling with or for hanging from an accessory pole 622, e.g., IV pole. Alternatively and/or additionally, the cable housing 612 may optionally incorporate a magnet 618 along the housing body for attachment to the accessory pole 622 or any other metallic surface.

The cable 620 may be extendable from the cable housing 612 and locked into place and cable housing 612 may also include an optional retraction toggle or switch 614 which allows for the retraction of the cable 620 back into the cable housing 612 or for the cable 620 to be set at a selected length. This allows for the cleaning device 610 to remain hanging from the accessory pole 622 such that the device 610 is readily available for use to a practitioner, e.g., for cleaning the port 632 of an IV tubing 630. The advancement mechanism 626 and cleaning element 628 may also be seen along the cleaning device 610.

FIG. 33C illustrates a perspective view of another variation where the cleaning device may be attached to a cable 620 retractably extending from a cable housing 636 which may be attached to the accessory pole 622 rather than to the body 461 of the cleaning device. The cable housing 636 may be attached through various mechanisms to any number of platforms.

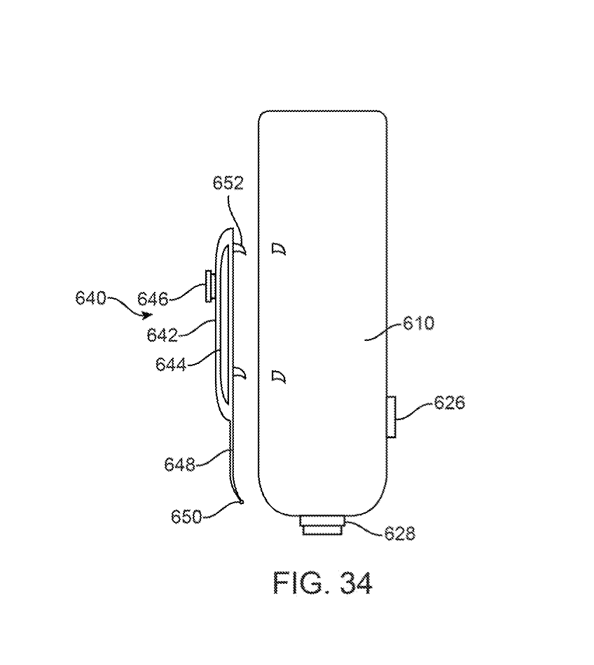

Another attachment option is illustrated in the side view of FIG. 34 which shows a fluid supply attachment 640 which may be attached to the cleaning device 610, e.g., via one or more connectors 652. The fluid supply attachment 640 may have a housing 642 which contains a fluid reservoir 644 and a fluid line 648 fluidly coupled to a fluid opening 650, e.g., atomizer. Such an attachment 640 may be included to optionally provide a cleaning or disinfecting or antiseptic fluid dispensed from the fluid reservoir 644 and through the fluid opening 650 to provide for another cleaning or disinfecting solution in addition to use of the cleaning element 628, if so desired.

Another attachment option may also include a fan or blower which may be removably attached to the cleaning device. The fan or blower may be used to speed up the drying of the cleaned surface or instrument.

Any of these attachments may be optionally incorporated with any of the device features and mechanisms described herein, as so desired.

Cleaning System

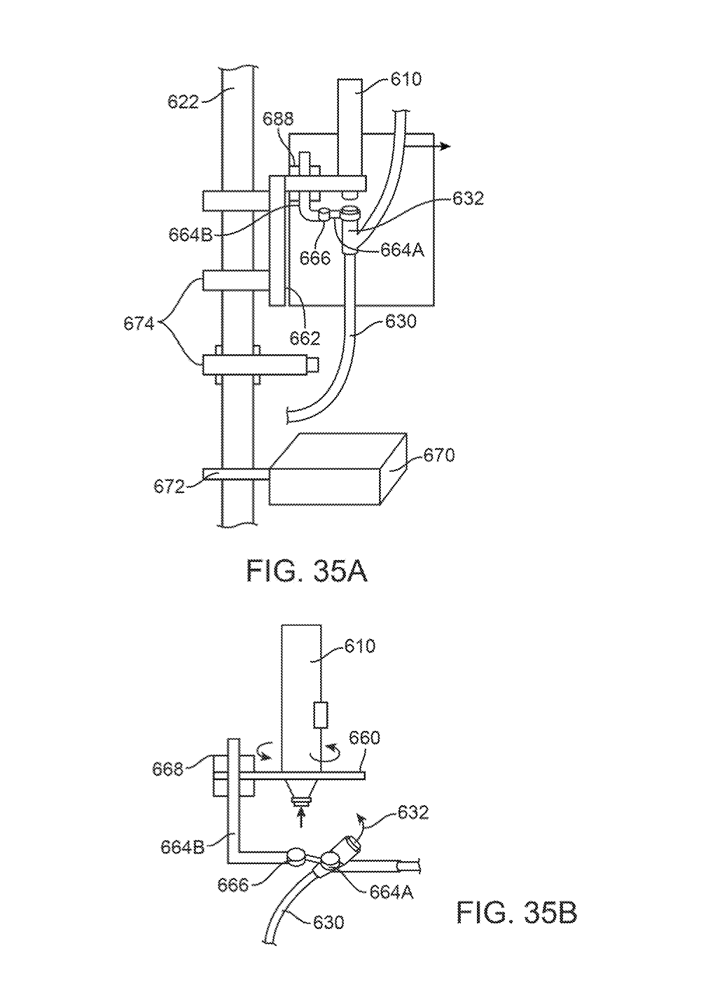

Because any of the cleaning device variations described and any of their combinations are portable and facilitate their use, the cleaning device may be used manually by the practitioner. Alternatively, use of the cleaning device may be automated to some degree. FIG. 35A illustrates a perspective view of one variation where the cleaning device 610 may be coupled to a platform 660 which may extend from a coupling arm 662 attachable to an accessory pole 622 or any other platform, e.g., via one or more clamps 674. The platform 660 may also have a first and second securing arm 664A, 664B extending from the platform 660 to a position below the platform 660. The first securing arm 664A may be connected to the second securing arm 664B via a pivot or hinge 666 which allows for the first securing arm 664A to be rotated or angled relative to the second securing arm 664B and platform 660.

A controller and motor 668 may be positioned along the platform 660 to provide of an optional rotation of the cleaning device 610 and/or providing for articulation of the first securing arm 664A about pivot or hinge 666. The first securing arm 664A may be pivoted relative to the platform 660 and cleaning device 610 until an instrument to be cleaned, e.g., port 632 is temporarily attached to the first securing arm 664A through any number of mechanisms such as a clamp. The controller and motor 668 may be activated to automatically pivot the first securing arm 664A and port 632 into alignment and contact with the cleaning device 610, as illustrated in the side view of FIG. 35B. The cleaning device 610 may then be configured to automatically activate its own actuator for cleaning the port 632 for a predetermined period of time, e.g., 1 to 15 seconds or more, after which the port 632 may be automatically lowered and pivoted for removal. The used cartridge may also be automatically ejected from the cleaning device 610 for disposal within a receptacle 670 positioned below the cleaning device 610. Also shown in an optional syringe cap dispenser 672 which may be attached to the accessory pole 622.

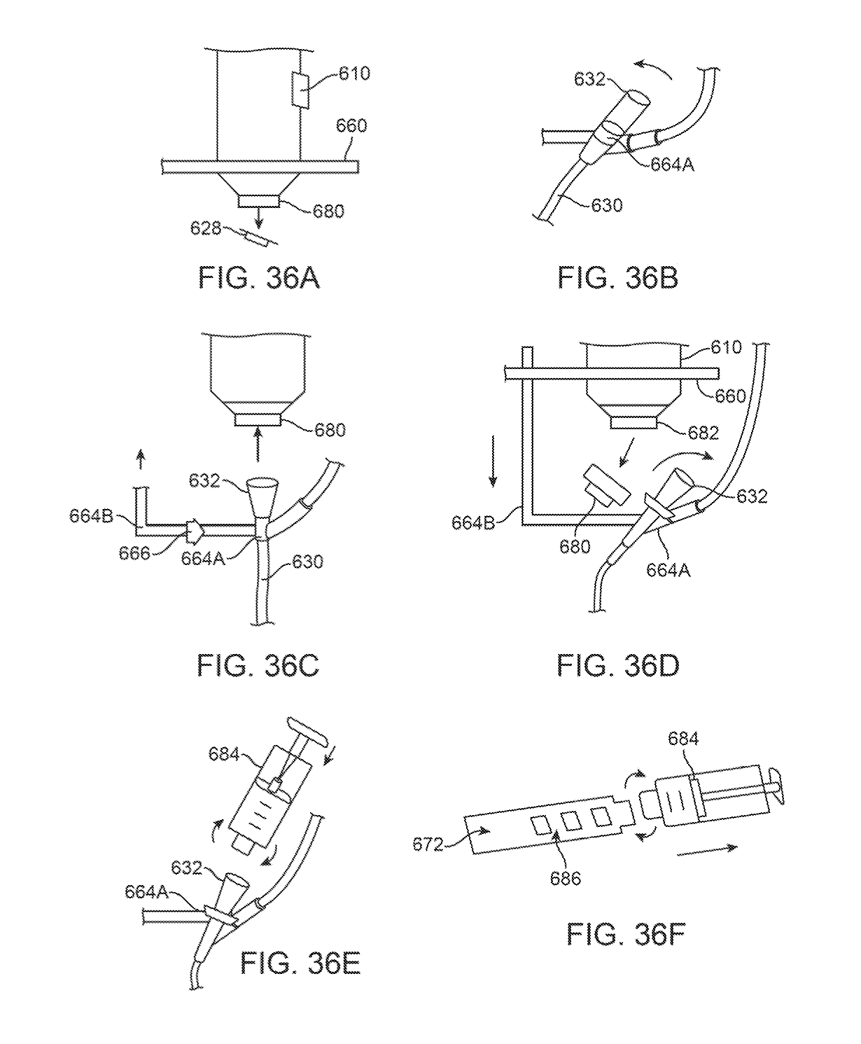

Another example is illustrated in FIGS. 36A to 36F which show a cleaning device 610 positioned along the platform. The cleaning device 610 may have a used cartridge 628 ejected and a new cartridge 680 advanced and secured along the distal opening of the device 610 for use, as shown in FIG. 36A. As previously described, the port 632 may be attached to the first securing aim 664A which may pivot, e.g., 45 degrees or other ergonomic angle, to align the port 632 beneath the cartridge 680 of the cleaning device 610, as shown in FIG. 36B. The port 632 may then be lifted via the controller and motor 668 moving the first and second securing arm 664A, 664B into contact against the cleaning element of cartridge 680, as shown in FIG. 36C. Once cleaning of the port 632 has been completed, the port 632 may be automatically or manually lowered from the cartridge 680 and the port 632 may be repositioned for removal from first securing arm 664A, as shown in FIG. 36D. The used cartridge 680 may then be ejected and a subsequent new cartridge 682 may be exposed for use.

A syringe 684 or other instrument may be optionally coupled to the port 632 while the port 632 remains secured to the first securing arm 664A all while avoiding any contact with the port 632, as shown in FIG. 36E. After the syringe 684 has been used, the optional syringe cap dispenser 672 may be used to dispense a syringe cap 686 on to the syringe 684, as shown in FIG. 36F, to seal the syringe 684 after use.

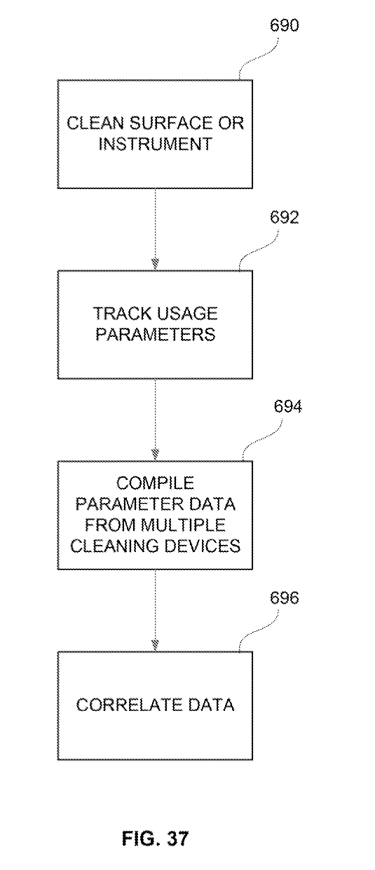

Furthermore, an optional data log may also be incorporated with the cleaning device. During use of the cleaning device, as shown in step 690 of FIG. 37, the controller and/or processor may be programmed to log information such as date and time for each use, as previously described and as shown in step 692. This information may be downloaded to a server system where it may be compiled and compared to the overall case statistics of a setting, e.g., hospital, to ensure that the cleaning device was used each time an instrument, such as a port, was accessed, as shown in step 694. The data log could be expanded and integrated deeper into the hospital system by logging location, user, and or patient and this tracking of information could increase accountability and better enable outcome tracking, as shown in step 696.

The cleaning device has tracking abilities, as described herein, where it can send the various recorded parameters to a particular electronic health record. The electronic tracking system can automatically determine if there are a series of mismatches between medication administration and device use which can be reported electronically back to the clinician or to an infection control professional. Another way the tracking system can be used is by finding an infection and then looking for the mismatch between medication administration and device use and thereby the offending clinician.

These cleaning systems are intended to be utilized with any of the cleaning device variations described herein and in any combination.

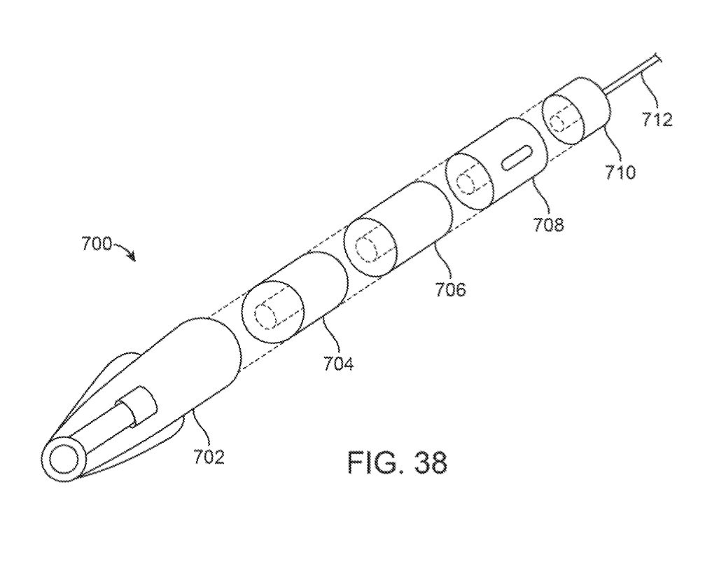

With all the various features described herein, the cleaning device may be configured to be assembled or built using modular components. For instance, FIG. 38 illustrates a perspective view of one variation of a cleaning device 700 which may be assembled using individual components which are each designed for a particular function (e.g., any of the various functions or features described herein). As shown, the cleaning device 700 may include a dispenser component 702 which may also include the actuator (e.g., motor) and processor or controller. The dispenser component 702 may include the power supply within or it may be incorporated into another component. An optional tracking component 704 may be electrically and mechanically coupled to the dispenser component 702 for optionally incorporating the processor or controller (if not included in the dispenser component 702) and the tracking component 704 may also include the power supply (e.g., disposable or rechargeable battery) if not included in the dispenser component 702.

An optional wireless transmitter and/or receiver component 706 having a wireless antenna for transmitting and/or receiving signals (e.g., radio-frequency, Bluetooth®, etc.) may be coupled proximally to the tracking component 704 and a scanner component 708 (e.g., two or three-dimensional barcode scanner) may be coupled to the component 706. An optional cable housing component 710 having a retractable cable 712 extendable therefrom may be coupled to the component 708. Collectively, each of the components may comprise the cleaning device 700.

While the variation shown in FIG. 38 includes each of the components described, other variations of the cleaning device 700 may omit certain components or may include other components not shown. Additionally, each of the components may be arranged in other configurations relative to one another and are not limited to the arrangement illustrated.

The applications of the devices and methods discussed above are not limited to the disinfection of TV ports, Luer fittings, skin surfaces, other surfaces, etc. but may include any number of further disinfection applications. Moreover, such devices and methods may be applied to other instruments or regions of the body to be disinfected. Modification of the above-described assemblies and methods for carrying out the invention, combinations between different variations as practicable, and variations of aspects of the invention that are obvious to those of skill in the art are intended to be within the scope of the claims.

What is claimed is:

1. A cleaning assembly, comprising:
an elongate housing defining a storage lumen therein and an opening at a distal end of the storage lumen;
one or more cartridges aligned adjacent to one another within the storage lumen with each cartridge having an alignment feature which translatably aligns the cartridges relative to the storage lumen, wherein each cartridge has a first surface and a second surface opposite to the first surface which has a cleaning element positioned upon and extending from the second surface such that the cleaning element from a proximal cartridge is positionable in proximity to the first surface of an adjacent distal cartridge;
a release mechanism positioned along the elongate housing and adjacent to the opening, wherein actuation of the release mechanism releases at least one cartridge positioned at the opening for ejection from the elongate housing;
an actuator within or along the elongate housing and in communication with the at least one cartridge positioned at the opening wherein activation of the actuator moves the cleaning element or the at least one cartridge relative to the elongate housing; and
a processor in electrical communication with the actuator, wherein the processor is programmed to monitor operation of the actuator and/or positioning of the one or more cartridges.

2. The assembly of claim 1 wherein the first surface defines a receiving cavity such that the cleaning element from the proximal cartridge is positionable within the receiving cavity of an adjacent distal cartridge.

3. The assembly of claim 1 further comprising a biasing mechanism configured to bias the one or more cartridges towards the opening at the distal end of the storage lumen.

4. The assembly of claim 1 wherein the cleaning element comprises an absorbent material.

5. The assembly of claim 4 wherein the cleaning element is comprised of an open, semi-open or semi-closed cell foams of different densities, or split microfiber.

6. The assembly of claim 1 wherein the cleaning element defines a cavity having a shape configured for receiving a port in a secure manner.

7. The assembly of claim 1 wherein the cleaning element comprises a center portion and an annular portion surrounding the center portion.

8. The assembly of claim 1 wherein the cleaning element extends beyond the opening of the cartridge.

9. The assembly of claim 1 wherein the cleaning element is negatively or positively charged.

10. The assembly of claim 1 further comprising a magnet or electromagnet in proximity to the cleaning element.

11. The assembly of claim 1 further comprising a sterilizing agent retained within the cleaning element.

12. The assembly of claim 11 wherein the sterilizing agent comprises isopropyl alcohol, chlorhexidine, hydrogen peroxide or silver, or combinations thereof.

13. The assembly of claim 12 further comprising an anesthetic agent retained within the cleaning element.

14. The assembly of claim 1 further comprising a seal removably secured upon the cartridge for retaining the cleaning element.

15. The assembly of claim 14 wherein the seal comprises a portion extending from a first end of the seal such that the portion is folded over the seal from the first end towards a second end of the seal which is opposite to the first end.

16. The assembly of claim 15 wherein distal translation of the cartridges towards the lumen opening engages a distal end of the portion such that the seal is pulled from the opening of the cartridge as the cartridges are advanced.

17. The assembly of claim 1 wherein the release mechanism comprises one or more release arm members which are biased to close upon the at least one cartridge positioned at the opening.

18. The assembly of claim 17 wherein the release arm members are further configured to release the at least one cartridge while retaining a remainder of the cartridges within the elongate housing when actuated.

19. The assembly of claim 1 wherein the actuator comprises a motor.

20. The assembly of claim 1 further comprising a biasing mechanism configured to push the one or more cartridges distally within the storage lumen.

21. The assembly of claim 1 wherein the processor is programmed to track and/or monitor usage information of the assembly.

22. The assembly of claim 21 wherein the processor is programmed to assign the cleaning assembly to a specified patient.

23. The assembly of claim 21 wherein the processor is further programmed to transmit the usage information to an electronic health record.

24. The assembly of claim 23 wherein the processor is in communication with a server remote from the elongate housing, wherein the server is programmed to compare a record of medication administration from the electronic health record against the usage information received from the processor and alert of a mismatch.

25. The assembly of claim 24 wherein the server is further programmed to monitor for a record of infection and to compare the record of medication administration against the usage information and alert of the mismatch.

26. The assembly of claim 1 wherein the processor is programmed to vary a time that the actuator is activated to actuate the cleaning element or the at least one cartridge based on a type of surface or instrument to be cleaned.

27. The assembly of claim 1 further comprising an adjustably retractable cable connectable to the elongate housing.

28. The assembly of claim 1 further comprising a fan connectable to the elongate housing.

29. The assembly of claim 1 further comprising a pressure sensor in communication with at least one cartridge and the processor, wherein a pressure applied to the at least one cartridge is sufficient to activate the actuator to move the cleaning element.

30. The assembly of claim 29 wherein the processor is further programmed to stop the actuator when the pressure is removed from the at least one cartridge.

31. The assembly of claim 30 wherein the processor is further programmed to eject the at least one cartridge when the pressure is removed from the at least one cartridge.

32. A method of disinfecting a region of interest with a cleaning assembly, comprising:
positioning a first cleaning element of a first cartridge in proximity to a distal opening of a storage lumen defined through an elongate housing;
activating an actuator which moves the cleaning element or the first cartridge relative to the elongate housing to clean the region of interest when contacted by the cleaning element;
actuating a release mechanism positioned along the elongate housing and adjacent to the opening such that the first cartridge is ejected from the elongate housing and a second cleaning element of a second cartridge is positioned within the opening; and
monitoring an operation of the cleaning assembly via a processor in communication with the actuator, wherein the processor is programmed to monitor positioning of the one or more cartridges.

33. The method of claim 32 wherein actuating a release mechanism further comprises pushing the first and second cartridges towards the opening at the distal end of the storage lumen.

34. The method of claim 32 wherein the cleaning element comprises an absorbent material.

35. The method of claim 34 wherein the cleaning element is comprised of an open, semi-open or semi-closed cell foams of different densities, or split microfiber.

36. The method of claim 32 wherein the cleaning element defines a cavity having a shape configured for receiving a port in a secure manner.

37. The method of claim 32 wherein the cleaning element comprises a center portion and an annular portion surrounding the center portion.

38. The method of claim 32 wherein the cleaning element extends beyond the opening of the cartridge.

39. The method of claim 32 wherein the cleaning element is negatively or positively charged.

40. The method of claim 32 further comprising a magnet or electromagnet in proximity to the cleaning element.

41. The method of claim 32 further comprising a sterilizing agent retained within the cleaning element.

42. The method of claim 41 wherein the sterilizing agent comprises isopropyl alcohol, chlorhexidine, hydrogen peroxide or silver, or combinations thereof.

43. The method of claim 42 further comprising an anesthetic agent.

44. The method of claim 32 wherein actuating a release mechanism further comprises removing a seal secured upon the second cartridge to expose the second cleaning element.

45. The method of claim 44 wherein distal translation of the second cartridge towards the opening engages a distal end of a tab such that the seal is pulled from an opening of the second cartridge.

46. The method of claim 32 wherein actuating a release mechanism comprises reconfiguring one or more release arm members from a closed configuration where the first cartridge is secured to an opened configuration where the first cartridge is unconstrained by the one or more release arm members.

47. The method of claim 32 wherein activating an actuator comprises moving the cleaning element via a motor.

48. The method of claim 32 wherein monitoring an operation comprises tracking and/or monitoring usage information of the assembly.

49. The method of claim 48 further comprising transmitting the usage information to an electronic health record.

50. The method of claim 49 wherein transmitting the usage information comprises transmitting to a server remote from the elongate housing.

51. The method of claim 50 further comprising comparing a record of medication administration from the electronic health record against the usage information received from the processor and alerting of a mismatch.

52. The method of claim 50 further comprising monitoring for a record of infection and comparing a record of medication administration against the usage information and alerting of a mismatch.

53. The method of claim 32 wherein activating an actuator further comprises detecting a pressure imparted upon the at least one cartridge via a pressure sensor and activating the actuator to move the cleaning element in response to the pressure.

54. The method of claim 53 further comprising stopping the actuator when the pressure is removed from the at least one cartridge.

55. The method of claim 54 further comprising actuating the release mechanism to eject the at least one cartridge when the pressure is removed from the at least one cartridge.

56. A method of disinfecting a portion of an instrument, comprising:
attaching the instrument to be cleaned to a securing arm which is positioned in an initial configuration in proximity to a platform;
actuating the securing arm to reposition the portion of the instrument to be cleaned from the initial configuration and into contact against a first cleaning element of a first cartridge positioned in proximity to a distal opening of a storage lumen defined through an elongate housing which is secured to the platform;
activating an actuator which moves the cleaning element or the first cartridge relative to the elongate housing and the platform to clean the portion of the instrument; and
repositioning the securing arm and the portion of the instrument back to the initial configuration after the portion of the instrument has been cleaned.

57. The method of claim 56 wherein attaching the instrument comprises attaching an IV port to the securing arm.

58. The method of claim 56 wherein actuating the securing arm comprises repositioning the portion from the initial configuration of 45 degrees relative to the platform.

59. The method of claim 56 wherein activating an actuator comprises automatically activating upon sensing a pressure imparted by the portion upon the cleaning element.

60. The method of claim 59 further comprising automatically moving the cleaning element for a predetermined period of time.

61. The method of claim 60 further comprising ejecting the first cartridge from the elongate housing after the predetermined period of time when the pressure is removed from the cleaning element,

62. A method of disinfecting a region of interest with a cleaning assembly, comprising:
positioning a first cleaning element of a first cartridge in proximity to a distal opening of a storage lumen defined through an elongate housing;
removing a seal from the first cartridge such that the first cleaning element is exposed at the distal opening;
activating an actuator which moves the first cleaning element or the first cartridge relative to the elongate housing to clean the region of interest when a pressure is imparted upon the first cleaning element;
stopping the actuator after a predetermined period of time has lapsed; and
ejecting the first cartridge from the distal when the pressure is removed from the first cleaning element and a second cleaning element of a second cartridge is positioned within the opening.

63. The method of claim 62 wherein removing a seal comprises automatically removing the seal from the first cartridge.

64. The method of claim 62 wherein activating an actuator comprises sensing the pressure via a pressure sensor in communication with a processor.

65. The method of claim 64 wherein the processor is programmed to stop the actuator after the predetermined period of time.

66. The method of claim 65 wherein the processor is programmed to eject the first cartridge when the pressure is no longer detected by the pressure sensor.

67. The method of claim 62 further comprising monitoring an operation of the cleaning assembly via a processor in communication with the actuator, wherein the processor is programmed to monitor positioning of the cartridges.

68. A cleaning assembly, comprising:
an elongate housing defining a storage lumen therein and an opening at a distal end of the storage lumen;
one or more cartridges aligned adjacent to one another within the storage lumen with each cartridge having an alignment feature which translatably aligns the cartridges relative to the storage lumen, wherein each cartridge has a first surface and a second surface opposite to the first surface which has a cleaning element positioned upon and extending from the second surface such that the cleaning element from a proximal cartridge is positionable in proximity to the first surface of an adjacent distal cartridge;
a release mechanism positioned along the elongate housing and adjacent to the opening, wherein actuation of the release mechanism releases at least one cartridge positioned at the opening for ejection from the elongate housing;
an actuator within or along the elongate housing and in communication with the at least one cartridge positioned at the opening wherein activation of the actuator moves the cleaning element or the at least one cartridge relative to the elongate housing;
a pressure sensor in communication with at least one cartridge and the processor, wherein a pressure applied to the at least one cartridge is sufficient to activate the actuator to move the cleaning element; and
a processor in electrical communication with the actuator, release mechanism, and pressure sensor.

69. The assembly of claim 68 wherein the first surface defines a receiving cavity such that the cleaning element from the proximal cartridge is positionable within the receiving cavity of an adjacent distal cartridge.

70. The assembly of claim 68 further comprising a seal removably secured upon the cartridge for retaining the cleaning element.

71. The assembly of claim 68 wherein the processor is programmed to track and/or monitor usage information of the assembly.

72. The assembly of claim 68 wherein the processor is programmed to assign the cleaning assembly to a specified patient.

73. The assembly of claim 68 wherein the processor is programmed to activate the actuator when the pressure sensor detects the pressure beyond a predetermined threshold level.

74. The assembly of claim 68 wherein the processor is programmed to stop the actuator when the pressure is removed from the at least one cartridge.

75. The assembly of claim 68 wherein the processor is programmed to eject the at least one cartridge when the pressure is removed from the at least one cartridge.

76. A cleaning device, comprising:
a dispenser component having one or more cartridges within, wherein the one or more cartridges are aligned adjacent to one another such that a cleaning element from a terminally positioned cartridge is positioned in proximity to a distal opening of the dispenser component;
a tracking component having a processor within which is in communication with the dispenser component, wherein the processor is programmed to monitor operation of the dispenser component;
a transmitter and/or receiver component having a wireless antenna in communication with the processor, wherein the transmitter and/or receiver is in wireless communication with a remote server; and
a scanner component in communication with the processor.

77. The device of claim 76 further comprising a cable housing component coupled to the device and having a cable projecting from the cable housing component.

78. The device of claim 76 wherein the dispenser component comprises an elongate housing defining a storage lumen therein such that the distal opening is located at a distal end of the storage lumen.

79. The device of claim 77 wherein the one or more cartridges each have an alignment feature which translatably aligns the cartridges relative to the storage lumen.

80. The device of claim 77 wherein each cartridge has a first surface and a second surface opposite to the first surface which has the cleaning element positioned upon and extending from the second surface such that the cleaning element from a proximal cartridge is positionable in proximity to the first surface of an adjacent distal cartridge.

81. The device of claim 76 wherein the dispenser component further comprises a release mechanism positioned adjacent to the distal opening, wherein actuation of the release mechanism releases at least one cartridge positioned at the opening for ejection from the dispenser component.

82. The device of claim 81 further comprising an actuator in communication with the at least one cartridge positioned at the opening wherein activation of the actuator moves the cleaning element or the at least one cartridge relative to the dispenser component.

83. The device of claim 82 wherein the processor is programmed to monitor operation of the actuator and/or positioning of the one or more cartridges.
